(19) United States
(12) Patent Application Publication
(10) Pub. No.: US 2005/0250123 A1
(43) Pub. Date: Nov. 10, 2005

(54) Reducing galectin-12 activity to reduce formation of adipocytes

(75) Inventors: Ri-Yao Yang, Davis, CA (US); Daniel K. Hsu, Davis, CA (US); Fu-tong Liu, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Regents of the University of California, Oakland, CA (US)

(21) Appl. No.: 10/995,793

(22) Filed: Nov. 22, 2004

Related U.S. Application Data
(60) Provisional application No. 60/524,418, filed on Nov. 21, 2003.

(51) Int. Cl. C12Q 1/68; C07H 21/02; A61K 48/00

(52) U.S. Cl. 435/6; 514/44; 536/23.1

(57) ABSTRACT

It has now been discovered that galectin-12 is necessary for the differentiation of pre-adipocytes into adipocytes and for differentiation of leukocytes. Inhibition of galectin-12 activity can therefore be used to block the formation of new fat cells or to down-regulate the formation of leukocytes, for example, to promote wound healing. The invention provides, for example, short, interfering RNAs (siRNAs) to inhibit expression of galectin-12 and its consequent activity. The invention further provides the use of inhibitors of galectin-12 activity and methods of inhibiting galectin-12 activity using such inhibitors.

### CROSS-REFERENCES TO RELATED APPLICATIONS

This application claims priority from and the benefit of U.S. Provisional Application No. 60/524,418, filed Nov. 21, 2004, the contents of which are incorporated by reference.

### STATEMENT AS TO RIGHTS TO INVENTIONS MADE UNDER FEDERALLY SPONSORED RESEARCH AND DEVELOPMENT

This invention was made with government support under grant numbers AI20958 and AI39620 awarded by the National Institute of Allergy and Infectious Diseases of the National Institutes of Health. The government has certain rights in the invention.

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Obesity is defined as a state of increased adipose tissue mass, of sufficient extent to produce adverse health consequences. Obesity is a major risk factor for non-insulin dependent diabetes mellitus (type 2 diabetes) and hypertension. It is also linked to some types of cancers and immune dysfunctions (Spiegelman, B. M. et al. J Biol. Chem. 268:6823-6826 (1993)). While the adipose tissue mass is contributed to by both the size and number of fat cells, the primary health concerns related to adipocyte development stem from extreme aberrations in fat cell number, which is accomplished by the differentiation of preadipocytes that act as the renewable source of adipocytes.

Much has been learnt about the transcriptional regulation of adipocyte differentiation in the past 20 years due to the availability of in vitro models of adipogenesis based on preadipocyte cell lines such as 3T3-L1 and 3T3-F422A. The major transcription factors in the transcription network of adipogenesis include CCAAT/enhancer-binding proteins, which belong to the basic-leucine zipper (bZIP) class of transcription factors (Ramji, D. P. et al. Biochem J. 365:561-575 (2002)), and peroxisome proliferators-activated receptor γ (PPARγ), a member of the nuclear hormone receptor superfamily (Rosen, E. D. et al. J Biol. Chem. 276:37731-37734 (2001)). Confluent 3T3-L1 preadipocytes can be differentiated into white adipocytes by treatment with the combination of insulin, a glucocorticoid, and an agent that elevates intracellular cyclic AMP levels, commonly abbreviated as MDI. C/EBPβ and δ are the first transcription factors induced after MDI treatment, which can be detected within 2 hours. After a long lag of about two days, they become competent to activate the expression of C/EBPα and PPARγ (Tang, Q. Q. et al., Genes Dev. 13:2231-2241 (1999)), which positively regulate each other's expression and mediate the transcription of a number of downstream genes that characterize the adipocyte phenotype (Rosen, E. D. et al. Genes Dev. 16:22-26 (2002)). The differentiation of brown adipocytes is regulated via a similar transcription network to that of white adipocytes. PPARγ and C/EBPα are induced during brown adipogenesis in a similar fashion to white adipocyte differentiation (Rosen, E. D. et al. Annu Rev Cell Dev Biol. 16:145-171 (2000)). The pivotal roles play by these transcription factors in adipogenesis have been demonstrated by both gain-of-function studies through ectopic expression in cell lines or mice, and loss-of-function studies that utilize antisense and knockout mice technologies (Rosen, E. D. et al. Genes Dev. 16:22-26 (2002); Rosen, E. D. et al. Mol Cell. 4:611-617 (1999); Tanaka, T. et al. EMBO J. 16:7432-7443 (1997); Wang, N. D. et al. Science, 269:1108-1112 (1995); Wu, Z. et al. Genes Dev. 9:2350-2363 (1995)).

Another protein believed to play an important role in adipogenesis is ADD1/SREBP1c, a member of the basic helix-loop-helix (bHLH) family of transcription factors, as suggested by its dramatic induction when cultured preadipocytic cell lines are stimulated to undergo differentiation (Kim, J. B. et al., Genes Dev. 10:1096-1107 (1996)). Although in vitro experiments demonstrated that ADD1/SREBP1c activity is required for adipogenesis, probably by generating some factors that enhances PPARγ activity (Kim, J. B. et al., Proc Natl Acad Sci USA. 95:4333-4337 (1998)), results from transgenic and knockout mice are not as conclusive (Shimano, H. et al. J Clin Invest. 100:2115-2124 (1997); Shimomura, I. et al. Genes Dev. 12:3182-3194 (1998)).

Galectins are a family of animal lectins with conserved carbohydrate-recognition domains (CRDs) for β-galactoside (Barondes, S. H. et al. J Biol. Chem. 269:20807-20810 (1994)). They are present in most species of the animal kingdom, including lower organisms, such as nematodes, and higher organisms, such as mammals. In mammals, fourteen members have been identified and more are likely to be discovered as more genomes are sequenced (Cooper, D. N. Biochim Biophys Acta. 1572:209-231 (2002)). The family can be subdivided into prototypical type (galectin-1, -2, -5, -7, -10, -13, and -14), which are monomers or homodimers of one carbohydrate-recognition domain (˜15 kDa); tandem repeat type (galectin-4, -6, -8, -9, and -12), which contain two distinct but homologous CRD in a single polypeptide chain; and chimeric type, where galectin-3 is the only member and contains a non-lectin part made of proline-, glycine-rich short tandem repeats connected to a CRD. Some of the members, especially galectin-3, which was first cloned from rat basophilic leukemic cells by this group by virtue of its binding to IgE (Liu, F. T. et al. Proc Natl Acad Sci USA. 82:4100-4104 (1985)), and galectin-1, have been extensively studied, and experimental results suggest that these lectins may have diverse functions (Liu, F. T. Clin Immunol. 97:79-88 (2000); Liu, F. T. et al. Biochim Biophys Acta. 1572:263-273 (2002)).

Most galectins have wide tissue distribution. Galectin-3, for example, is abundantly present in the epithelia of several organs (Liu, F. T. Clin Immunol. 97:79-88 (2000)), as well as in various inflammatory cells, including monocytes/macrophages (Liu, F. T. et al. Am J Pathol. 147:1016-1028 (1995)). Consistent with the lack of a classical signal sequence, galectins are mainly intracellular proteins (Liu, F. T. Clin Immunol. 97:79-88 (2000)). However, a number of studies have demonstrated the secretion of these proteins. The mechanism underlying the secretion of galectins is not well understood, but plasma membrane targeting and vesicular budding are thought to be critically involved in the secretion of galectin-3 (Mehul, B. et al. J Cell Sci. 110(10):1169-1178 (1997)). More recently, galectin-3 has been identified as a component of exosomes in dendritic cells (Thery, C. et al., J Immunol. 166:7309-7318 (2001)), suggesting an interesting possibility that this lectin and other galectins are secreted as a part of exosomes. Consistent with their spatial distribution, these proteins appear to function both intracellularly and extracellularly. The extracellular functions are likely to be due to carbohydrate-binding properties and in many cases are inhibited by specific free carbohydrate, while the intracellular functions may not be related to carbohydrate binding (Liu, F. T. et al. Biochim Biophys Acta. 1572:263-273 (2002)). Although all galectins contain at least one homologous CRD in their sequence, different members exhibit different localization and expression patterns, suggesting distinct functions for each member of the family (Lowe, J. B. Cell, 104:809-812 (2001); Rabinovich, G. A. et al., Trends Immunol. 23:313-320 (2002); Yang, R. Y. et al. Cell Mol Life Sci. 60:267-276 (2003)).

Galectin-12 is a galectin with two CRDs. The N-terminal CRD is highly homologous to those of other galectins, while its C-terminal CRD shows significant divergence (Yang, R. Y. et al., J Biol. Chem. 276:20252-20260 (2001) (hereafter, “Yang 2001”)). Its mRNA contains AU-rich motifs in the 3′-untranslated region, and the initiation codon for translation locates in a suboptimal context (Yang 2001), suggesting vigorous post-transcriptional regulation at the levels of mRNA stability (Chen, C. Y. et al., Trends Biochem Sci. 20:465-470 (1995)) and translation efficiency (Kozak, M. Gene, 299:1-34 (2002)). The expression of this gene is very restricted, with high expression only in adipocytes (Hotta, K. et al. J Biol. Chem. 276:34089-34097 (2001) (hereafter, “Hotta 2001”)) and peripheral blood leucocytes (Yang 2001). Galectin-12 is up-regulated when cells are blocked at the G1 phase and ectopic expression of this protein causes cell cycle arrest at the G1 phase with concomitant cell growth suppression (Yang 2001). Its expression in adipocytes is down-regulated by agents known to impair insulin sensitivity, implying a role for galectin-12 in the pathogenesis of type 2 diabetes (Fasshauer, M. et al. Eur J Endocrinol. 147:553-559 (2002)).

Like the differentiation of many other cell lineages, adipocyte differentiation is interwoven with changes in cell cycle status culminating in the permanent exit from the cell cycle (Reichert, M. et al. Oncogene, 18:459-466 (1999)). Cell cycle arrest before adipogenic hormone treatment is required for the initiation of subsequent differentiation events, and permanent cell cycle exit afterwards is essential for the completion of the differentiation process (Gregoire, F. M. et al. Physiol Rev. 78:783-809 (1998)). It is therefore not surprising that many proteins with cell cycle regulatory functions, such as Rb (Cowherd, R. M. et al. Semin Cell Dev Biol. 10:3-10 (1999)), c-Myc (Freytag, S. O. et al. Science, 256:379-382 (1992)), and E2Fs (Fajas, L. et al. Dev Cell. 3:39-49 (2002)), also have potent effects on adipocyte differentiation. Reciprocally, the two master regulators of adipocyte differentiation, C/EBPα (Porse, B. T. et al. Cell, 107:247-258 (2001); Umek, R. M. et al. Science, 251:288-292 (1991)) and PPARγ (Altiok, S. et al. Genes Dev. 11:1987-1998 (1997)), both possess the ability to cause G1 arrest.

“RNA interference”, a form of post-transcriptional gene silencing (“PTGS”), describes effects that result from the introduction of double-stranded RNA into cells (reviewed in Fire, A. Trends Genet 15:358-363 (1999); Sharp, P. Genes Dev 13:139-141 (1999); Hunter, C., Curr Biol 9:R440-R442 (1999); Baulcombe. D., Curr Biol 9:R599-R601 (1999); Vaucheret et al., Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo. In mammalian cells other than these, however, longer RNA duplexes provoked a response known as “sequence non-specific RNA interference,” characterized by the non-specific inhibition of protein synthesis.

Further studies showed this effect to be induced by dsRNA of greater than about 30 base pairs, apparently due to an interferon response. It is thought that dsRNA of greater than about 30 base pairs binds and activates the protein PKR and 2′,5′-oligonucleotide synthetase (2′,5′-AS). Activated PKR stalls translation by phosphorylation of the translation initiation factors eIF2α, and activated 2′,5′-AS causes mRNA degradation by 2′,5′-oligonucleotide-activated ribonuclease L. These responses are intrinsically sequence-nonspecific to the inducing dsRNA; they also frequently result in apoptosis, or cell death. Thus, most somatic mammalian cells undergo apoptosis when exposed to the concentrations of dsRNA that induce RNAi in lower eukaryotic cells.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes are too short to elicit sequence-nonspecific responses like apoptosis, yet they efficiently initiate RNAi. Many laboratories rushed to have siRNA made to knock out target genes in mammalian cells. The results demonstrate that siRNA works quite well in most instances, far better and more consistently than do ribozymes, antisense or other nucleic acid agents.

### BRIEF SUMMARY OF THE INVENTION

In a first group of embodiments, the invention provides short interfering (si) RNAs comprising a first and a second strand, each strand (a) being of equal length, (b) from 16 to 29 nucleotides in length, (c) hybridized to the other strand, and (d) having a 5′ end and a 3′ end, and wherein in which the 16 to 29 nucleotides of one strand are complementary to a corresponding length of galectin-12 mRNA (SEQ ID NO:1). In some uses, at least 1 nucleotide, but not more than 4 nucleotides, at a 5′ end of a strand is deoxyribose nucleic acid. In some uses, the invention provides a siRNA in which at least 1 nucleotide, but not more than 4 nucleotides, at a 3′ end of a strand is deoxyribose nucleic acid. The siRNAs may further comprise at least 1 unpaired nucleotide at the 3′ end of each strand. At least one unpaired nucleotide at the 3′ end of at least one strand may be a deoxyribose nucleic acid. In some uses, one of the two strands of the siRNA has a sequence selected from the group consisting of SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30. Any of these sequences may further comprise at least 1 unpaired nucleotide at the 3′ end of each strand. Further, any of these sequences may have at least 1 deoxyribose nucleic acid nucleotide, but not more than 4 such nucleotides, at the 5′ end of the sequence. The first and the second strand of the siRNA may be selected from the group consisting of (a) SEQ ID NOS:3 and 4, (b) SEQ ID NOS:5 and 6, (c) SEQ ID NOS:7 and 8, (d) SEQ ID NOS:9 and 10, (e) SEQ ID NOS:11 and 12, (f) SEQ ID NOS:13 and 14, (g) SEQ ID NOS:15 and 16, (h) SEQ ID NOS:31 and 32, (i) SEQ ID NOS:33 and 34, (j) SEQ ID NOS:35 and 36, (k) SEQ ID NOS:37 and 38, (l) SEQ ID NOS:39 and 40, (m) SEQ ID NOS:41 and 42, (n) SEQ ID NOS:43 and 44, (o) SEQ ID NOS:45 and 46, (p) SEQ ID NOS:47 and 48, (q) SEQ ID NOS:49 and 50, (r) SEQ ID NOS:51 and 52, (s) SEQ ID NOS:53 and 54, (t) SEQ ID NOS:55 and 56, (u) SEQ ID NOS:57 and 58, (v) SEQ ID NOS:59 and 60, and (w) SEQ ID NOS:61 and 62. The first and second strands of the siRNA, including siRNA having the sequences set forth above, may be linked by a spacer to permit formation of a hairpin configuration.

In a second group of embodiments, the invention provides vectors encoding the siRNAs described above. More specifically, the invention vectors comprising a first promoter operably linked to a nucleic acid comprising a first segment that encodes at least a first strand of a short interfering (si) RNA from 16 to 29 nucleotides in length, which said strand has a 5′ end and a 3′ end, in which said 16 to 29 nucleotides of said first strand are complementary to a corresponding length of galectin-12 mRNA (SEQ ID NO:1). The vector may further comprise a second segment that encodes a RNA complementary to that encoded by said first segment. The vector may further comprise a second segment that encodes a RNA complementary to that encoded by said first segment and a linker between the first segment and the second segment. The vector may also comprise a second promoter positioned to permit transcription of RNA in a direction antiparallel to the first promoter and which, when transcribed antiparallel to said the promoter, results in transcription of a RNA complementary to the first strand. The said siRNA may further comprise at least 1 unpaired nucleotide at the 3′ end of the first strand. The first strand may, for example, have a sequence selected from the group consisting of SEQ ID NO:9, SEQ ID NO:10, SEQ ID NO:11, SEQ ID NO:12, SEQ ID NO:13, SEQ ID NO:14, SEQ ID NO:21, SEQ ID NO:22, SEQ ID NO:23, SEQ ID NO:24, SEQ ID NO:25, and SEQ ID NO:26.

In yet another set of embodiments, the invention provides use of a short, interfering (si) RNA from 16 to 29 nucleotides in length, the siRNA having a first strand and a second strand, each strand being (a) of equal length, (b) from 16 to 29 nucleotides in length, (c) hybridized to the other strand, and (d) having a 5′ end and a 3′ end, and wherein in which the 16 to 29 nucleotides of one strand are complementary to a corresponding length of galectin-12 mRNA (SEQ ID NO:1), or a vector encoding such a siRNA, for manufacture of a medicament to inhibit differentiation of pre-adipocytes. The siRNA may further comprise at least 1 unpaired nucleotide at the 3′ end of each strand. The strands may, for example, be selected from the group consisting of (a) SEQ ID NOS:3 and 4, (b) SEQ ID NOS:5 and 6, (c) SEQ ID NOS:7 and 8, (d) SEQ ID NOS:9 and 10, (e) SEQ ID NOS:11 and 12, (f) SEQ ID NOS:13 and 14, (g) SEQ ID NOS:15 and 16, (h) SEQ ID NOS:31 and 32, (i) SEQ ID NOS:33 and 34, (j) SEQ ID NOS:35 and 36, (k) SEQ ID NOS:37 and 38, (l) SEQ ID NOS:39 and 40, (m) SEQ ID NOS:41 and 42, (n) SEQ ID NOS:43 and 44, (o) SEQ ID NOS:45 and 46, (p) SEQ ID NOS:47 and 48, (q) SEQ ID NOS:49 and 50, (r) SEQ ID NOS:51 and 52, (s) SEQ ID NOS:53 and 54, (t) SEQ ID NOS:55 and 56, (u) SEQ ID NOS:57 and 58, (v) SEQ ID NOS:59 and 60, and (w) SEQ ID NOS:61 and 62.

The invention also provides the use of an inhibitor of galectin-12 activity for manufacture of a medicament to inhibit differentiation of pre-adipocytes. Further, the invention provides the use of an inhibitor of galectin-12 activity for manufacture of a medicament to inhibit differentiation of leukocytes.

In still a further set of embodiments, the invention provides compositions comprising a short, interfering (si) RNA from 16 to 29 nucleotides in length, the siRNA having a first strand and a second strand, each strand being (a) of equal length, (b) from 16 to 29 nucleotides in length, (c) hybridized to the other strand, and (d) having a 5′ end and a 3′ end, and wherein in which the 16 to 29 nucleotides of one strand are complementary to a corresponding length of galectin-12 mRNA (SEQ ID NO:1), and a pharmaceutically acceptable carrier. The siRNA may further comprise at least 1 unpaired nucleotide at the 3′ end of each strand. At least one unpaired nucleotide at the 3′ end of at least one strand may be a deoxyribose nucleic acid. At least 1 nucleotide, but not more than 4 nucleotides, at a 5′ end of a strand is deoxyribose nucleic acid. At least 1 nucleotide, but not more than 4 nucleotides, at a 3′ end of a strand may be deoxyribose nucleic acid. In some embodiments, one of the two strands has a sequence selected from the group consisting of SEQ ID NO:9, SEQ ID NO:10, SEQ ID NO:11, SEQ ID NO:12, SEQ ID NO:13, SEQ ID NO:14, SEQ ID NO:21, SEQ ID NO:22, SEQ ID NO:23, SEQ ID NO:24, SEQ ID NO:25, and SEQ ID NO:26. In other embodiments, the strands are selected from the group consisting of (a) SEQ ID NOS:3 and 4, (b) SEQ ID NOS:5 and 6, (c) SEQ ID NOS:7 and 8, (d) SEQ ID NOS:9 and 10, (e) SEQ ID NOS:11 and 12, (f) SEQ ID NOS:13 and 14, (g) SEQ ID NOS:15 and 16, (h) SEQ ID NOS:31 and 32, (i) SEQ ID NOS:33 and 34, (j) SEQ ID NOS:35 and 36, (k) SEQ ID NOS:37 and 38, (l) SEQ ID NOS:39 and 40, (m) SEQ ID NOS:41 and 42, (n) SEQ ID NOS:43 and 44, (o) SEQ ID NOS:45 and 46, (p) SEQ ID NOS:47 and 48, (q) SEQ ID NOS:49 and 50, (r) SEQ ID NOS:51 and 52, (s) SEQ ID NOS:53 and 54, (t) SEQ ID NOS:55 and 56, (u) SEQ ID NOS:57 and 58, (v) SEQ ID NOS:59 and 60, and (w) SEQ ID NOS:61 and 62.

Moreover, the invention provides methods of inhibiting differentiation of a pre-adipocyte to an adipocyte. The method comprises contacting a pre-adipocyte with (i) a short, interfering (si) RNA from 16 to 29 nucleotides in length, the siRNA having a first strand and a second strand, each strand being (a) of equal length, (b) from 16 to 29 nucleotides in length, (c) hybridized to the other strand, and (d) having a 5′ end and a 3′ end, and wherein in which the 16 to 29 nucleotides of one strand are complementary to a corresponding length of galectin-12 mRNA (SEQ ID NO:1), (ii) a vector encoding a siRNA of (i), or (iii) both (i) and (ii), thereby inhibiting activity of galectin-12 in the pre-adipocyte. In some uses, the first strand and the second strand have at least one unpaired nucleotide on their respective 3′ ends. In some uses, one of the two strands has a sequence selected from the group consisting of SEQ ID NO:9, SEQ ID NO:10, SEQ ID NO:11, SEQ ID NO:12, SEQ ID NO:13, SEQ ID NO:14, SEQ ID NO:21, SEQ ID NO:22, SEQ ID NO:23, SEQ ID NO:24, SEQ ID NO:25, and SEQ ID NO:26. In other uses, the strands are selected from the group consisting of (a) SEQ ID NOS:3 and 4, (b) SEQ ID NOS:5 and 6, (c) SEQ ID NOS:7 and 8, (d) SEQ ID NOS:9 and 10, (e) SEQ ID NOS:11 and 12, (f) SEQ ID NOS:13 and 14, (g) SEQ ID NOS:15 and 16, (h) SEQ ID NOS:31 and 32, (i) SEQ ID NOS:33 and 34, (j) SEQ ID NOS:35 and 36, (k) SEQ ID NOS:37 and 38, (l) SEQ ID NOS:39 and 40, (m) SEQ ID NOS:41 and 42, (n) SEQ ID NOS:43 and 44, (o) SEQ ID NOS:45 and 46, (p) SEQ ID NOS:47 and 48, (q) SEQ ID NOS:49 and 50, (r) SEQ ID NOS:51 and 52, (s) SEQ ID NOS:53 and 54, (t) SEQ ID NOS:55 and 56, (u) SEQ ID NOS:57 and 58, (v) SEQ ID NOS:59 and 60, and (w) SEQ ID NOS:61 and 62.

The invention further provides methods of inhibiting differentiation of pre-adipocytes into adipocytes, comprising administering an inhibitor of galectin-12 activity, thereby inhibiting the differentiation of pre-adipocytes.

Additionally, the invention provides methods of inhibiting differentiation of leukocytes, comprising administering an inhibitor of galectin-12 activity, thereby inhibiting said differentiation of leukocytes.

In yet a further group of embodiments, the invention provides kits comprising (a) a container and (b) (i) a short, interfering (si) RNA from 16 to 29 nucleotides in length, said siRNA having a first strand and a second strand, each strand being (A) of equal length, (B) from 16 to 29 nucleotides in length, (C) hybridized to the other strand, and (D) having a 5′ end and a 3′ end, and wherein in which the 16 to 29 nucleotides of one strand are complementary to a corresponding length of galectin-12 mRNA (SEQ ID NO:1), or (ii) a vector encoding a siRNA of (b)(i), or (iii) both (b)(i) and (b)(ii). The kit may further comprise a pharmaceutically acceptable carrier.

### BRIEF DESCRIPTION OF THE DRAWINGS

FIGS. 1a and 1b. Mouse galectin-12 cDNA Sequence. The major open reading frame is shown against a grey background. CA repeats are underlined and the two AT-rich motifs in the 3′-UTR are boxed. These sequence data are available from GenBank/EMBL/DDBJ under accession no. AF223223. b. Exon organization of mouse and human galectin-12 genes. Exons shown in black code for two CRDs in galectin-12 polypeptides. Numbers indicate in base pairs (bp) the length of each exon and intron. Note that all corresponding exons in human and mouse galectin-12 genes are of the same lengths.

FIG. 2. Protein sequence comparison of mouse and human galectin-12 with other 2-CRD galectins. Sequences corresponding to amino acid residues 161-186 in galectin-12 comprise the linker regions. The comparison was generated with the program PILEUP in the GCG package and shadings are used to indicate degrees of similarities.

FIGS. 3a-3d. Galectin-12 expression during adipocyte differentiation of mouse 3T3-L1 cells correlates with the cell cycle status. Total RNA or protein was extracted from subconfluent and confluent cells, as well as from cells harvested at different time points after adipogenic hormone treatment. Gene expression was analyzed by RT-PCR with primers specific for galectin-12 or G3PDH as control (a), or by Western blotting with antibodies for galectin-12 or a-tubulin (b). (c): Subconfluent, confluent, and differentiating 3T3-L1 cells at various time points after adipogenic hormone stimulation were harvested and fixed with 70% ethanol. Nuclei were stained with propidium iodide and cell cycle distribution (DNA content) was determined by flow cytometry. (d): The first and the second peaks in the DNA histogram represent cells in the G1/G0 and the G2/M phase, respectively, while those with DNA content in between are S phase cells. The extent of G1 arrest is reflected by the G1/S ratio as shown by the number in each histogram.

FIGS. 4a-4c. Down-regulation of endogenous galectin-12 expression in mouse 3T3-L1 preadipocytes. a. Sequences of siRNAs used in the experiments. b. Positions of sequences in mouse galectin-12 mRNA targeted by the 3 siRNAs. c. Western blotting showing the decreased galectin-12 protein levels 3 days after transfection with galectin-12 siRNAs.

FIG. 5a-5d. Effects of galectin-12 knockdown on adipocyte differentiation. Subconfluent 3T3-L1 cells were transfected with indicated siRNAs (FIG. 4). Three days later, cells were subjected to the pro-differentiative regimen to induce adipocyte differentiation. Ten days after adipogenic treatment, cells were stained with the lipophilic dye Oil Red O to assess adipocyte differentiation, as indicated by the accumulation of lipid droplets (red) in the cytoplasm. Nuclei were counterstained with SYBR Green-1 and are shown in green in this merged confocal image (a). Quantification of Oil Red O staining was achieved after solubilizing the dye with isopropanol by measuring the absorbance at 510 nm (b). Expression of insulin receptor and its substrate IRS-1 in cells transfected with indicated siRNAs were measured 10 days after adipogenic treatment by Western blotting with antibodies to insulin receptor P subunit (c) or IRS-1 (d).

FIGS. 6a-6b. Expression of adipogenic transcription factors are defective in galectin-12 knockdown cells. a, Three days after siRNA transfection (Time 0), pro-adipogenic hormonal cocktail was added and protein extracted at different time points for Western blotting to detect C/EBPβ induction. b, Five days after adipogenic hormone treatment, expression of C/EBPα and PPARγ, the two key adipogenic factors downstream of C/EBPβ, was determined by Western blotting with respective antibodies. The two bands correspond to two major translation products of C/EBPα and C/EBPβ produced using alternative translation initiation sites (Calkhoven, C. F. et al. Genes Dev. 14:1920-1932 (2000)). No changes in galectin-3 expression were seen in galectin-12 knockdown cells (b). Equal amount of total protein (10 μg) was loaded in each lane.

FIGS. 7a-7c. Galectin-12 is required for optimal activation of ERK (a) and Akt (b) by adipogenic hormones, but not for IFG-1 receptor autophosphorylation (c). Three days after transfection with indicated siRNAs, 3T3-L1 cells were treated with the adipogenic hormone cocktail for various lengths of time before harvested for Western blotting with antibodies to ERK or Akt, or their respective phosphorylated forms. Ten μg of proteins were loaded per lane. Tyrosine phosphorylation of IGF-1 receptor was measured by immunoprecipitation with anti-phosphotyrosine antibody PY20 and Western blotting with an antibody to IGF-1 receptor (c).

### DETAILED DESCRIPTION OF THE INVENTION

Introduction

A. Reduction of Galectin-12 Expression in Pre-Adipocytes Blocks Differentiation into Adipocytes

Work from the laboratory of the present inventors has previously resulted in the discovery of galectin-12, a member of a family of proteins with highly conserved carbohydrate binding domains that bind α-galactosides. Yang et al. J Biol Chem 276(33):20252-20260 (2001). Shortly thereafter, Hotta et al., J Biol Chem 276(36):34089-34097 (2001), reported that galectin-12 was expressed in adipose tissue. Hotta et al. reported that galectin-12 had apoptosis-inducing properties and suggested that the protein might play an important role in the reduction of fat mass through inducing apoptosis of adipocytes. Id. Thus, based on Hotta et al., it might be expected that reducing the expression of galectin-12 would result in maintaining fat deposition, and would likely result in increased fat deposits.

Surprisingly, it has now been found that reducing expression of galectin-12 in pre-adipocytes inhibits the differentiation of the pre-adipocytes into adipocytes. Far from maintaining fat deposition, therefore, the present discovery shows that reduction of expression of galectin-12 can be used to avoid the formation of new fat cells, and therefore of increased fat deposits.

It has also been discovered that galectin-12 is also involved in leukocyte differentiation. Reduction of galectin-12 activity can be used to reduce this differentiation. It is known that the numbers of leukocytes need to drop to promote efficient wound healing. Reducing galectin-12 activity after injuries resulting in wounds promotes more rapid and complete healing of the wound. The practitioner will typically administer compositions which reduce galectin-12 activity after ensuring that any infections which might have been introduced with the injuries that resulted in the wounds are cleared.

In a preferred set of embodiments, galectin-12 activity is reduced by reducing expression of galectin-12 in pre-adipocytes or lymphocytes. In a particularly preferred set of embodiments, galectin-12 activity is reduced by the use of small interfering (“si”) RNAs which specifically inhibit expression of galectin-12.

B. siRNAs of the Invention

In a preferred embodiment, galectin-12 expression can be reduced by the use of siRNA. A program for predicting siRNA, siDESIGN from Dharmacon, Inc. (Lafayette, Colo.), available on the World Wide Web at dharmacon.com, predicts from the mRNA sequence (SEQ ID NO:1) for human galectin-12 that the following sequences of RNA will function as siRNA:

5′-GAUAUCGCCUUCCACUUCAUU-3′(SEQ ID NO:3)
3′-UUCUAUAGCGGAAGGUGAAGU-5′(SEQ ID NO:4)

Other programs for selecting siRNAs to inhibit galectin-12 are available on the World Web Web at, for example, ambion.com/techlib/misc/siRNA_finder.html and by entering “http://” followed by “jura.wi.mit.edu/pubint/http://iona.wi.mit.edu/siRNAext/”.

Additionally, the present inventors have demonstrated that siRNA can be used to reduce galectin-12 expression in mouse cells, targeting regions of mouse mRNA for mouse galectin-12 mRNA that are share 100% identity with human galectin-12 mRNA. Given the complete conservation of identity of mRNA between the species in this region, it is expected that the siRNA sequences shown to reduce galectin-12 expression in mouse cells will likewise work in reducing expression of galectin-12 in human cells. These siRNAs are

5′-UUCCUGAACAUCAAUCCAUUU-3′(SEQ ID NO:5)
3′-UUAAGGACUUGUAGUUAGGUA-5′(SEQ ID NO:6)
and
5′-CAUCAAUCCAUUUGUGGAGUU-3′(SEQ ID NO:7)
3′-UUGUAGUUAGGUAAACACCUC-5′(SEQ ID NO:8)

Including the 3′ untranslated regions and using less stringent criteria resulted in the design of the following sequences:

| | |
|---|---|
| 5′-CAAGAGUGCAAAGGUUCCUUU-3′ | (SEQ ID NO:9) |
| 3′-UUGUUCUCACGUUUCCAAGGA-5′, | (SEQ ID NO:10) |
| 5′-GAGGCAAGUGUUGUAGACUUU-3′ | (SEQ ID NO:11) |
| 3′-UUCUCCGUUCACAACAUCUGA-5′ | (SEQ ID NO:12) |
| 5′-GGCAAGUGUUGUAGACUAAUU-3′ | (SEQ ID NO:13) |
| 3′-UUCCGUUCACAACAUCUGAUU-5′ | (SEQ ID NO:14) |
| 5′-AUACAAUGGCUUAAAGAAUUU-3′ | (SEQ ID NO:15) |
| 3′-UUUAUGUUACCGAAUUUCUUA-5′ | (SEQ ID NO:16) |

The exemplar nucleic acid pairs above have been offset to emphasize that they include unpaired nucleotides, termed “overhangs”, on the 3′ end of the sequences. Thus, the nucleic acids of the invention preferably have at least one unpaired nucleotide on the 3′ end. The nucleic acids may have up to 15 unpaired nucleotides on the 3′ end, but more preferably have 14, 13, 12, 11, 10, 9, 8, 7, 6, 5, 4, or 3 unpaired nucleotides, with each successive smaller number being more preferred. Studies have shown that 21 nucleotide siRNA duplexes are most active when containing two-nucleotide 3′-overhangs. Thus, nucleic acids with two unpaired nucleotides on the 3′ end are most preferred. The overhangs are preferably of uridines. Nucleotides other than U can be used, however, it is preferred that the overhang not include a G because the siRNA may be cleaved by RNase at single-stranded G nucleotides.

Persons of skill will recognize, however, that the overhangs, while preferred for maximum activity, are not absolutely required and that the same sequences can be utilized to inhibit galectin-12 without employing overhangs. Thus, for example, the sequences set forth above can instead be presented as:

| | |
|---|---|
| 5′-GAUAUCGCCUUCCACUUCA-3′ | (SEQ ID NO:17) |
| 3′-CUAUAGCGGAAGGUGAAGU-5′ | (SEQ ID NO:18) |
| 5′-UUCCUGAACAUCAAUCCAU-3′ | (SEQ ID NO:19) |
| 3′-AAGGACUUGUAGUUAGGUA-5′ | (SEQ ID NO:20) |
| 5′-CAUCAAUCCAUUUGUGGAG-3′ | (SEQ ID NO:21) |
| 3′-GUAGUUAGGUAAACACCUC-5′ | (SEQ ID NO:22) |
| 5′-CAAGAGUGCAAAGGUUCCU-3′ | (SEQ ID NO:23) |
| 3′-GUUCUCACGUUUCCAAGGA-5′, | (SEQ ID NO:24) |
| 5′-GAGGCAAGUGUUGUAGACU-3′ | (SEQ ID NO:25) |
| 3′-CUCCGUUCACAACAUCUGA-5′ | (SEQ ID NO:26) |
| 5′-GGCAAGUGUUGUAGACUAA-3′ | (SEQ ID NO:27) |
| 3′-CCGUUCACAACAUCUGAUU-5′ | (SEQ ID NO:28) |
| and | |
| 5′-AUACAAUGGCUUAAAGAAU-3′ | (SEQ ID NO:29) |
| 3′-UAUGUUACCGAAUUUCUUA-5′ | (SEQ ID NO:30) |

Studies have shown that replacing the 3′-overhanging segments of a siRNA duplex having 2 nucleotide 3′ overhangs with deoxyribonucleotides does not have an adverse effect on RNAi activity. Thus, the above sequences can have deoxyribose nucleotides, preferably thymidines, in place of the uridines shown in the sequences above, or one deoxyribose nucleotide and one ribose nucleotide (such as a UT combination). Thus, for example, the sequences set forth above can have deoxyribose nucleotide overhangs, such as:

| | |
|---|---|
| 5′-GAUAUCGCCUUCCACUUCATT-3′ | (SEQ ID NO:31) |
| 3′-TTCUAUAGCGGAAGGUGAAGU-5′ | (SEQ ID NO:32) |
| 5′-UUCCUGAACAUCAAUCCAUTT-3′ | (SEQ ID NO:33) |
| 3′-TTAAGGACUUGUAGUUAGGUA-5′; | (SEQ ID NO:34) |
| and, | |
| 5′-CAUCAAUCCAUUUGUGGAGTT-3′ | (SEQ ID NO:35) |
| 3′-TTGUAGUUAGGUAAACACCUC-5′ | (SEQ ID NO:36) |

Replacing up to 4 nucleotides on each end of the siRNA with deoxyribonucleotides has been reported to be well tolerated whereas complete substitution with deoxyribonucleotides results in no RNAi activity (Elbashir et al., 2001, supra). Thus, 1, 2, 3, or 4 nucleotides on the 5′ or 3′ end, or both, of the siRNA sequences of the invention, such as those set forth above, can be deoxyribonucleotides. For ease of reference, such molecules, which comprise both RNA and DNA, are referred to herein as “siRNA” unless otherwise required by context.

Studies have also indicated that the position of the cleavage site in the target RNA is defined by the 5′-end of the siRNA guide sequence rather than the 3′-end (Elbashir et al., EMBO J., 20:6877 (2001)). Other studies have indicated that a 5′-phosphate on the target-complementary strand of a siRNA duplex is required for siRNA activity and that ATP is utilized to maintain the 5′-phosphate moiety on the siRNA (Nykanen et al., Cell, 107:309 (2001)).

Persons of skill in the art are aware that ds RNAs shorter than 30 nucleotides do not induce sequence non-specific RNA interference. Moreover, some studies have indicated that the inhibition caused by less active 19-mer duplex siRNAs can be increased if the length of the duplexed RNA is increased, while the activity of more active 19-mer duplexes is not affected. See, e.g., Yu et al., Mol Ther 7(2):228-36 (2003). Persons of skill will therefore recognize that siRNA duplexes up to 29 nucleotides in length can be designed to with regard to the same target portion of galectin-12 mRNA as those impacted by the 19-nucleotide duplexes set forth above by extending the target-complementary strand to complement the sequence of galectin-12 mRNA (SEQ ID NO:1). Thus, siRNAs of 20, 21, 22, 23, 24, 25, 26 27, 28 or 29 nucleotide duplexes can be created from the sequences set forth above and SEQ ID NO: 1. Examples of some of these longer duplexes are:

(SEQ ID NO:37)

5′-GAUAUCGCCUUCCACUUCAACCCUCGCUUUU-3′

(SEQ ID NO:38)

3′-UUCUAUAGCGGAAGGUGAAGUUGGGAGCGAA-5′

(SEQ ID NO:39)

5′-UUCCUGAACAUCAAUCCAUUUGUGGAGGGUU-3′

(SEQ ID NO:40)

3′-UUAAGGACUUGUAGUUAGGUAAACACCUCCC-5′

(SEQ ID NO:41)

5′-CAUCAAUCCAUUUGUGGAGGGCAGCAGAGUU-3′

(SEQ ID NO:42)

3′-UUGUAGUUAGGUAAACACCUCCCGUCGUCUC-5′

(SEQ ID NO:43)

5′-GAUAUCGCCUUCCACUUCAACCCUCGCUUU-3′

(SEQ ID NO:44)

3′-UUCUAUAGCGGAAGGUGAAGUUGGGAGCGA-5′

(SEQ ID NO:45)

5′-UUCCUGAACAUCAAUCCAUUUGUGGAUU-3′

(SEQ ID NO:46)

3′-UUAAGGACUUGUAGUUAGGUAAACACCU-5′

(SEQ ID NO:47)

5′-CAUCAAUCCAUUUGUGGAGGGCAGCUU-3′

(SEQ ID NO:48)

3′-UUGUAGUUAGGUAAACACCUCCCGUCG-5′

(SEQ ID NO:49)

5′-GAUAUCGCCUUCCACUUCAACUU-3′

(SEQ ID NO:50)

3′-UUCUAUAGCGGAAGGUGAAGUUG-5′

(SEQ ID NO:51)

5′-UUCCUGAACAUCAAUCCAUUUGUU-3′

(SEQ ID NO:52)

3′-UUAAGGACUUGUAGUUAGGUAAAC-5′

(SEQ ID NO:53)

5′-CAUCAAUCCAUUUGUGGAGGGCUU-3′

(SEQ ID NO:54)

3′-UUGUAGUUAGGUAAACACCUCCCG-5′

(The two-nucleotide overhangs, of course, are not counted when determining the length of the duplex since they are not paired.)

Further, while 19-nucleotide duplexes are considered a preferred embodiment, duplexes of 16, 17, or 18 nucleotides in length are also known to function as siRNAs and can be used in the methods of the invention. The 19-nucleotide duplexes of the exemplar sequences set forth above can be shortened by one, two or three nucleotides (preferably by omitting the nucleotides at the 3′ end of the sequences shown in 5′ to 3′ orientation, and the corresponding nucleotides of the complementary sequence). Thus, for example, SEQ ID NOS:3 and 4 can be shortened to create the following:

| | |
|---|---|
| 18-mer duplex form: | |
| 5′-GAUAUCGCCUUCCACUUC-3′ | (SEQ ID NO:55) |
| 3′-CUAUAGCGGAAGGUGAAG-5′; | (SEQ ID NO:56) |
| 17-mer duplex form: | |
| 5′-GAUAUCGCCUUCCACUU-3′ | (SEQ ID NO:57) |
| 3′-CUAUAGCGGAAGGUGAA-5′ | (SEQ ID NO:58) |
| 16-mer duplex form: | |
| 5′-GAUAUCGCCUUCCACU-3′ | (SEQ ID NO:59) |
| 3′-CUAUAGCGGAAGGUGA-5′. | (SEQ ID NO:60) |

Each of these duplex forms will preferably be generated with 3′ overhangs to enhance potency, for example:

| | |
|---|---|
| 18-mer duplex form: | |
| 5′-GAUAUCGCCUUCCACUUCUU-3′ | (SEQ ID NO:61) |
| 3′-UUCUAUAGCGGAAGGUGAAG-5′; | (SEQ ID NO:62) |

Similar shortened forms can, of course, be formed from the other exemplar sequences set forth above. And, of course, each of these duplexes can have deoxyribose nucleotides as overhangs, or deoxyribose nucleotides as the first 1-4 nucleotides of the duplex sequence, or both. Thus, each of the sequences set forth above represents both the initial sequence and a cluster of permitted variants.

While siRNAs of the invention can be designed as set forth above, it will be appreciated that siRNAs can also be generated from galectin-12 mRNA without designing individual siRNAs. For example, the “Dicer siRNA Generation” kit (catalog number T510001, Gene Therapy Systems, Inc., San Diego, Calif.) uses recombinant human dicer enzyme in vitro to cleave long double stranded RNA into 22 bp siRNAs. By having a mixture of siRNAs, the kit permits a high degree of success in generating siRNAs that will reduce expression of the target gene. Similarly, the Silencer™ siRNA Cocktail Kit (RNase III) (catalog no. 1625, Ambion, Inc., Austin, Tex.) generates a mixture of siRNAs from dsRNA using RNase III instead of dicer. Like dicer, RNase III cleaves dsRNA into 12-30 bp dsRNA fragments with 2 to 3 nucleotide 3′ overhangs, and 5′-phosphate and 3′-hydroxyl termini. According to the manufacturer, dsRNA is produced using T7 RNA polymerase, and reaction and purification components included in the kit. The dsRNA is then digested by RNase III to create a population of siRNAs. The kit includes reagents to synthesize long dsRNAs by in vitro transcription and to digest those dsRNAs into siRNA-like molecules using RNase III. The manufacturer indicates that the user need only supply a DNA template with opposing T7 phage polymerase promoters or two separate templates with promoters on opposite ends of the region to be transcribed.

The siRNAs of the invention can also be expressed from vectors. Typically, such vectors are administered in conjunction with a second vector encoding the corresponding complementary strand. Once expressed, the two strands anneal to each other and form the functional double stranded siRNA. In other embodiments, the vector contains two promoters, one positioned downstream of the first and in antiparallel orientation. The first promoter is transcribed in one direction, and the second in the direction antiparallel to the first, resulting in expression of the complementary strands. In yet another set of embodiments, the promoter is followed by a first segment encoding the first strand, and a second segment encoding the second strand. The second strand is complementary to the palindrome of the first strand. Between the first and the second strands is a section of RNA serving as a linker (sometimes called a “spacer”) to permit the second strand to bend around and anneal to the first strand, in a configuration known as a “hairpin.”

The formation of hairpin RNAs, including use of linker sections, is well known in the art. Typically, an siRNA expression cassette is employed, using a Polymerase III promoter such as human U6, mouse U6, or human H1. The coding sequence is typically a 19-nucleotide sense siRNA sequence linked to its reverse complementary antisense siRNA sequence by a short spacer. Nine-nucleotide spacers are typical, although other spacers can be designed. For example, the Ambion website indicates that its scientists have had success with the spacer TTCAAGAGA. Further, 5-6 T's are often added to the 3′ end of the oligonucleotide to serve as a termination site for Polymerase III. See also, Yu et al., Mol Ther 7(2):228-36 (2003); Matsukura et al., Nucleic Acids Res 31(15):e77 (2003);

C. Uses of the Invention

The studies reported herein show that siRNA to galectin-12 inhibits the differentiation of pre-adipocytes to adipocytes. This affords a number of uses.

In vitro, inhibitors of galectin-12 activity, such as siRNAs targeting galectin-12 mRNA, can be used as a reagent to reduce the differentiation of pre-adipocytes into adipocytes, even when the pre-adipocytes are exposed to growth factors that would normally induce differentiation. This permits dissection and study of the cellular processes induced by the growth factors.

In vivo, inhibitors of galectin-12 activity, such as siRNAs targeting galectin-12 mRNA, can be used in several contexts. In situations in which a subject might undergo weight gain over a fairly short interval, such as an “all-you-can-eat” cruise, a trip to Paris, or the approach of the Thanksgiving and Christmas holidays, inhibitors of galectin-12 activity, such as siRNA, can be administered to avoid adding fat during the period. For example, administration of vectors encoding siRNA to galectin-12 that would result in short-term or transient expression of the siRNA can be used to reduce galectin-12 expression during some or all of the period in question, resulting in a reduction in fat deposition during the period. It is anticipated that inhibitors of galectin-12 activity for such short term administration, such as siRNA, can be administered systemically or locally.

There are also situations in which longer term administration of inhibitors of galectin-12 activity is desirable. For example, a person who is morbidly obese faces serious health consequences, and some 300,000 Americans currently die annually from obesity-related diseases. Current medical approaches to reducing morbid obesity often involve extensive surgery and have significant life-altering consequences. For example, one surgical approach, gastric bypass, popularly known as “stomach-stapling,” not only removes a significant part of the patient's stomach, but also leaves the individual unable to eat more than a few ounces of food at a time, rendering it difficult for the individual to obtain adequate nutrition.

Thus, any risk to the individual patient which might be incurred by long term administration of inhibitors of galectin-12 activity, such as siRNA targeted to galectin-12 mRNA, must be balanced against (a) the risk to the individual of leaving the obesity untreated and (b) the risk to the individual of the surgical alternatives. Further, long term administration of siRNA to galectin-12 can serve as an adjunct to other treatments, such as surgical methods. Use of siRNA as an adjunct may permit use of less aggressive surgical methods, with consequent improvement of the patient's post-surgical quality of life.

Methods for administration of inhibitors of galectin-12 activity, such as siRNA, are discussed in sections below. In general, however, in short-term administration (for example, where the siRNA will be expressed transiently), the inhibitors of galectin-12 activity can be administered either systemically or locally. For longer term administration, it may be desirable to administer the inhibitors of galectin-12 activity, such as siRNA, locally to reduce any impact of the inhibitor on peripheral blood leukocytes.

Reduction of leukocytes is, however, advantageous in promoting rapid healing of wounds. Thus, once the wound is debrided and cleaned and any infections consequent to an injury have been cleared (once, for example, the injured individual is in the hospital and antibiotics administered), inhibitors of galectin-12, such as siRNAs, can be used to reduce leukocyte differentiation, thereby promoting rapid healing of the injury. Similarly, the inhibitors can be used after surgery to promote rapid healing of the surgical incision. Since myeloid progenitors reside in the bone marrow, inhibition of leukocyte differentiation is preferably conducted by systemic administration of inhibitors of galectin-12 activity.

Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Galectin-12” is a member of a family of animal lectins with conserved carbohydrate-recognition domains (CRDs) for β-galactoside. Galectin-12 has two CRDs and, unlike other galectins, has very limited expression in human tissues. Galectin-12 has sequenced, as reported by Yang, R. Y. et al. J Biol. Chem. 276:20252-20260 (2001) and Strausberg et al. PNAS 99(26):16899-16903 (2002). The mRNA sequence (SEQ ID NO:1) and amino acid sequence (SEQ ID NO:2) are available in the Entrez browser of the National Center for Biotechnology Information, which can be accessed on the World Wide Web at “ncbi.nlm.nih.gov/entrez.” The mRNA nucleotide sequence (SEQ ID NO:1) is available under accession numbers NM_{—}033101 and BC028222 on the Entrez nucleotide browser; the translated amino acid sequence (SEQ ID NO:2) encoded by the mRNA for galectin-12 is available at those accessions and under accession numbers AAH28222 and NP_{—}149092 on the Entrez protein browser.

“Pre-adipocytes” are stromal cells capable of differentiating into adipose cells. Typically, pre-adipose cells express the β-1 adrenoceptor and β-2 adrenoceptor, but not the β-3 adrenoreceptor, and do not express the VEGF receptor. The cells can be isolated from subcutaneous adipose tissue by known techniques, such as those disclosed in Hauner H, et al., J. Clin. Invest. 84:1663-1670 (1989); Marko et al., Endocrinology 136:4582-4588 (1994); and Halvorsen et al., Metabolism 50(4):407-413 (2001). In culture, pre-adipocytes in culture have a fibroblast-like appearance and are immediately responsive to lipogenic hormones. Techniques for differentiating pre-adipocytes into adipocytes are also known in the art and are taught in, e.g., Hauner, supra, and Marko, supra. Human pre-adipocytes are commercially available from, for example, BioCat GmbH (Heidelberg, Germany).

“Adipocytes” are fat cells. In culture, they are rounded and filled with lipid droplets, rendering them easily identified. In the body, adipose tissue (comprising large numbers of adipocytes) is also easily identified by the practitioner. For example, tens of thousands of elective liposuction surgeries to remove excess adipose tissue are performed annually. Human adipocytes are commercially available from, for example, BioCat GmbH (Heidelberg, Germany).

“dsRNA” and “dsRNA molecule” refer to an RNA molecule comprising two complementary RNA strands hybridized together through base pairing interactions.

The term “siRNAs” refers to short interfering RNAs. In some embodiments, siRNAs comprise a duplex, or double-stranded region, where each strand of the double stranded region is about 18 to about 25 nucleotides long; the double stranded region can be as short as 16, and as long as 29, base pairs long, where the length is determined by the antisense strand. Often, siRNAs contain from about two to four unpaired nucleotides, known as “overhangs,” at the 3′ end of each strand. Often, the overhanging nucleotides are uridines. The nucleotides constituting the overhang can be deoxyribose nucleic acids, as can the first 1, 2, 3, or 4 nucleotides on the 5′ or 3′ ends, or both of the siRNA sequences of the invention, such as those set forth above, can be deoxyribonucleotides. For ease of reference, such molecules, which comprise both RNA and DNA, are referred to herein as “siRNA” unless otherwise required by context. Functionally, the characteristic distinguishing an siRNA over other forms of dsRNA is that the siRNA comprises a sequence capable of specifically inhibiting genetic expression of a target gene or closely related family of genes by a process termed “RNA interference.”

At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. One strand of the double stranded region need not be the exact length of the opposite strand; thus, one strand may have at least one fewer nucleotides than the opposite complementary strand, resulting in a “bubble” or at least one unmatched base in the opposite strand. One strand of the double stranded region need not be exactly complementary to the opposite strand; thus, the strand, preferably the sense strand, may have at least one mismatched base-pair.

siRNAs may also contain additional sequences; non-limiting examples of such sequences include linking sequences, or loops, which connect the two strands of the duplex region. This form of siRNAs may be referred to “si-like RNA,” “short hairpin siRNA,” where the short refers to the duplex region of the siRNA, or “hairpin siRNA.” Additional non-limiting examples of additional sequences present in siRNAs include stem and other folded structures. The additional sequences may or may not have known functions; non-limiting examples of such functions include increasing stability of an siRNA molecule, or providing a cellular destination signal.

The term “target RNA molecule” refers to an RNA molecule to which at least one strand of the short double-stranded region of an siRNA is complementary. Typically, when such complementarity is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

The term “ds siRNA” refers to a siRNA molecule which comprises two separate unlinked strands of RNA which form a duplex structure, such that the siRNA molecule comprises two RNA polynucleotides.

The term “hairpin siRNA” refers to a siRNA molecule which comprises at least one duplex region where the strands of the duplex are connected or contiguous at one or both ends, such that the siRNA molecule comprises a single RNA polynucleotide. The antisense sequence, or sequence which is complementary to a target RNA, comprises at least a part of the double stranded region.

The term “RNA interference” or “RNAi” refers to the silencing or decreasing expression, or inhibition of expression, of gene expression by siRNAs. It is the process of sequence-specific, post-transcriptional gene silencing in animals and plants initiated by siRNA that is homologous in its duplex region to the sequence of the silenced gene or that is complementary in its duplex region to the transcriptional product of the silenced gene. The gene may be endogenous or exogenous to the organism, present integrated into a chromosome or present in a transfection vector which is not integrated into the genome. The expression of the silenced gene is either completely or partially inhibited.

The term “sequence-nonspecific gene silencing” refers to silencing gene expression in mammalian cells after transcription, and is induced by dsRNA of greater than about 30 base pairs. This appears to be due to an interferon response, in which dsRNA of greater than about 30 base pairs binds and activates the protein PKR and 2′,5′-oligonucleotide synthetase (2′,5′-AS). Activated PKR stalls translation by phosphorylation of the translation initiation factors eIF2alpha, and activated 2′,5′-AS causes mRNA degradation by 2′,5′-oligonucleotide-activated ribonuclease L. These responses are intrinsically sequence-nonspecific to the inducing dsRNA.

The term “annealing” refers to the process of cooling a solution of nucleic acids comprising complementary sequences, in such a manner as to allow the base pairs of the complementary strands to bond together through Watson-Crick base pairing.

The terms “5′ primer” and “3′ primer” refer to short nucleic acid molecules having sequences complementary to the 5′ and 3′ ends, respectively, of a nucleic acid larger than either primer and in many cases, larger than the combined length of both the 5′ and 3′ primers. The term “blocking primers” refers to a pair of 5′ and 3′ primers that are complementary to the 5′ and 3′ ends, respectively, of a nucleic acid larger than the combined length of both the 5′ and 3′ primers.

The term “bases” refers to the individual nucleotides making up a polynucleotide.

The term “cell population” generally refers to a grouping of cells of a common type, typically having a common progenitor, although the phrase is also applicable to heterogeneous cell populations.

The term “cell division” refers to the physical cellular event, and preceding biochemical events, that culminate in a cell splitting into two autonomous units.

The term “cellular growth” refers to those cellular processes that lead to an increase in cell mass, volume, or number.

The term “cellular gene” or “gene” refers to a nucleic acid fragment that encodes a specific transcription product and includes regulatory sequences preceding (5′ non-coding) and following (3′ non-coding) the coding region that control transcriptional expression.

The term “cell genome” refers to the endogenous genetic material of a cell, and any exogenous genetic material that has been inserted into or substituted for the endogenous genetic material.

The term “cell surface marker” refers to any biological molecule associated with the outer surface of a cell membrane and detectable either physically or chemically.

The terms “complementary” or “complementarity” refer to polynucleotides (i.e., a sequence of nucleotides) related by base-pairing rules. For example, the sequence “5′-AGT-3′,” is complementary to the sequence “5′-ACT-3′”. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance for methods that depend upon binding between nucleic acids.

A “complementary termination sequence” refers to a nucleic acid sequence that has a nucleotide sequence complementary to a transcription termination sequence of a given promoter.

The term “operably linked” refers to a linkage of polynucleotide elements in a functional relationship. With regard to the present invention, the term “operably linked” refers to a functional linkage between a nucleic acid expression control sequence (such as a promoter, or an array of transcription factor binding sites) and a second nucleic acid sequence, wherein the expression control sequence directs transcription of the nucleic acid corresponding to the second sequence. Thus, a nucleic acid is “operably linked” when it is placed into a functional relationship with another nucleic acid sequence.

In relation to proteins, the term “denaturing” refers to a loss of secondary or tertiary structure of a protein molecule. In relation to double-stranded nucleic acids, denaturing refers to the dissociation of previously base-paired polynucleotides, either partially or fully, into two separate polynucleotide strands.

The terms “detectable marker”, “detectable trait” and “detectable cellular trait” refer to any physical or chemical characteristic expressed by a cell that can be identified by observation or test.

A “DNA expression cassette” or, simply, “expression cassette” refers to a DNA sequence capable of directing expression of a nucleic acid in cells. A “DNA expression cassette” comprises a promoter, operably linked to a nucleic acid of interest, which is further operably linked to a termination sequence. In the case of linear DNA expression cassettes, the termination sequence can be omitted if the 3′ end of the coding sequence is located at the end of the molecule. In this case, “termination” occurs when the RNA polymerase runs off the end of the molecule.

The term “exogenous” refers to any molecule or agent that is foreign to its current environment, as in originating, being derived or developing from a source other than the current environment.

The phrase “eukaryotic cell population” refers to one or more cells characterized by having their genomic DNA encased in a nuclear envelope or membrane when in “S” phase of the mitotic cycle.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the recombinant expression cassette portion of the expression vector includes a nucleic acid to be transcribed, and a promoter.

The term “extracellular protein” refers to any material, at least partially proteinaceous in character, located outside of a cell.

The term “fluorescent protein” refers to any material, at least partially proteinaceous in character, capable of emitting fluorescent energy in response to excitation by electromagnetic energy.

The term “gene expression” refers to all processes involved in producing a biologically active agent, whether nucleic acid or protein, from a nucleic acid encoding the biologically active agent. Gene expression includes all post-transcriptional and/or post-translational processing required to produce the mature agent.

The term “host cell” refers to a cell that contains an expression vector and supports the replication or expression of the expression vector. A host cell can be prokaryotic cells such as E. coli, or eukaryotic cells such as yeast, insect, or mammalian cells.

“Inducible” means that a promoter sequence, and hence the nucleic acid sequence whose expression it controls, is subject to regulation in response to factors which act as “inducers”. These factors can be proteins, nucleic acids, small molecules or physical stimuli e.g. UV irradiation. Induction of regulated nucleic acid sequences may involve the binding of factors that directly stimulate activity, or alternatively may require the removal of factors so as to derepress expression of a nucleic acid sequence. Induction can be measured, for example by treating cells with a potential inducer and comparing the expression of a nucleic acid sequence in the induced cells to the activity of the same nucleic acid sequence in control samples not treated with the inducer. Control samples (untreated with inducers) are assigned a relative activity value of 100%. Induction of a nucleic acid sequence is achieved when the activity value relative to the control (untreated with inducers) is 110%, more preferably 150%, more preferably 200-500% (i.e., two to five fold higher relative to the control), more preferably 1000-3000% higher.

The phrase “inhibiting expression of a cellular gene by the siRNA” refers to sequence-specific inhibition of genetic expression by a small interfering RNA molecule (siRNA) characterized by degradation of specific mRNA(s). The process is also referred to as RNA interference or RNAi.

The term “Klenow polymerase” is the polymerase activity remaining after treatment of E. coli DNA polymerase I with the protease subtilisin to separate the 5′3′ exonuclease activity of the holoenzyme.

In the context of this invention, the term “ligate” and its grammatic derivatives, refers to a covalent attachment of one molecule to another. For example, two polynucleotides are said to be ligated when the 5′ end of one is covalently bound to the 3′ end of the other.

The term “nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof.

The term “nucleic acid sequence” refers to the particular placement of nucleotide bases in relation to each other as they appear in a polynucleotide.

Promoters, terminators and control elements “operably linked” to a nucleic acid sequence of interest are capable of effecting the expression of the nucleic acid sequence of interest. The control elements need not be contiguous with the coding sequence, so long as they function to direct the expression thereof. Thus, for example, a promoter or terminator is “operably linked” to a coding sequence if it affects the transcription of the coding sequence.

The term “operator sequence” refers to a DNA sequence recognized by a specific protein or nucleic acid, that upon binding inhibits or prevents transcription from an adjacent operator sequence. An example is the tetracycline (tet) operator/repressor system.

The term “phenotypic change” refers to any change in physical, morphologic, biochemical or behavioral characteristics of a cell that can be identified by observation or test.

The term “phenotypic difference” refers to an expressed genetically-based difference in physical, morphologic, biochemical or behavioral characteristics between two or more cells or organisms of the same strain or species.

A “promoter” refers to an array of nucleic acid control sequences that direct transcription of a nucleic acid. As used herein, a promoter includes necessary nucleic acid sequences near the start site of transcription, such as, in the case of a type III RNA polymerase III promoter, a TATA element. A promoter also optionally includes proximal and distal sequence elements, which can be located as much as several hundred base pairs from the start site of transcription. A “constitutive” promoter is a promoter that is active under most environmental and developmental conditions. An “inducible” promoter is a promoter that is active under environmental or developmental regulation. Thus, the term “promoter” means a nucleotide sequence that, when operably linked to a DNA sequence of interest, promotes transcription of that DNA sequence.

The term “promoter region” refers to a nucleotide region comprising a DNA regulatory sequence, wherein the regulatory sequence is derived from a gene which is capable of binding an RNA polymerase and initiating transcription a given nucleic acid sequence. The “promoter region” of a given gene or set of genes, determines which of the three eukaryotic RNA polymerases will enjoy the task of transcribing that gene or nucleic acid sequence. The present invention is primarily concerned with genes and nucleic acid sequences transcribed by eukaryotic RNA polymerase III.

Eukaryotic RNA polymerase III transcribes a limited set of genes comprising 5SRNA, tRNA, 7SL RNA, U6 snRNA and a few other small stable RNAs. To function efficiently, most RNA polymerase III promoters require sequence elements downstream of the +1 transcription start site, within the transcribed region. However, type III RNA polymerase III promoters, do not require any intragenic sequence elements to function. Instead, efficient expression from type III RNA polymerase III promoters depends on the presence of upstream sequence elements comprising; a TATA box between −30 and −24, a proximal sequence element (PSE) between −66 and −47, and, in some cases, a distal sequence element (DSE) between −265 and −149. The best characterized type III RNA polymerase III promoters are those associated with the human H1 RNA and U6 snRNA genes.

The term “randomized” or “randomized sequence”, when referring to any nucleic acid sequence, indicates that the nucleotide base appearing at any given position in the sequence said to be randomized can be any one of the five nucleotides occurring naturally in RNA and DNA, or any homologue thereof, such that a complete set of randomized nucleic acids for a given length will consist of members having every base sequence permutation over the given length. The randomized sequences can be totally randomized (i.e., the probability of finding a base at any position being one in four) or only partially randomized (e.g., the probability of finding a base at any location can be selected at any level between 0 and 100 percent).

Nucleic acid sequence variants can be produced in a number of ways including chemical synthesis of randomized nucleic acid sequences and size selection from randomly cleaved cellular nucleic acids. Usually, the random nucleic acids are chemically synthesized so that the sequences may incorporate any nucleotide at any position. However, if it is desirable to do so, a bias may be deliberately introduced into the randomized sequence, for example, by altering the molar ratios of precursor nucleoside (or deoxynucleoside) triphosphates of the synthesis reaction. A deliberate bias may be desired, for example, to approximate the proportions of individual bases in a given organism, or to affect secondary structure. Thus, the randomized nucleic acid sequence may contain a fully or partially randomized sequence; or it may contain subportions of conserved sequence incorporated with randomized sequence. Thus, the synthetic process can be designed to allow the formation of any possible combination over the length of the sequence, thereby forming a library of randomized candidate nucleic acids.

The phrase “partially randomized nucleic acid sequence” refers to a nucleic acid sequence consisting of both randomized and predetermined sequences. The randomized portion of the sequence is completely randomized, as described herein above. The predetermined portion of the sequence is known to the user of the invention prior to synthesis of the partially randomized sequence. Predetermined sequences are predominantly included to ease cloning and synthesis of complementary nucleic acid strands, as described herein.

The term “restriction site” refers to a DNA sequence that can be recognized and cut by a specific restriction enzyme.

The terms “segment” or “sequence segment” refer to portions of nucleic acids and sequences of the same, the sequence segment being a subsequence of a larger nucleic acid. Typically, segments will possess functional characteristics, for example regulation of genetic expression, or form a coding sequence or structural domain of the nucleic acid. In the case of coding segments, the segment may encode a structural and or functional feature of the encoded molecule.

“Signal transduction” refers to a process by which the information contained in an extracellular physical or chemical signal (e.g., hormone or growth factor) is received by the cell by the activation of specific receptors and conveyed across the plasma membrane, and along an intracellular chain of various components, to stimulate the appropriate cellular response.

“Signal transduction pathway components,” “pathway components,” or “components of a signal transduction pathway” refer to intracellular or transmembrane biomolecules (of a particular apparent molecular weight) which are activated in cascade in response to an extracellular signal received by the cell.

The term “signal transduction pathway” refers to those biochemical events whereby a chemical or physical event impinging upon a cell is transmitted to a cellular process leading to a change in the physical or metabolic state of the cell in response to the original chemical or physical event.

The term “self-replicating” refers to a genetic element possessing one or more independent replication origins that function within a cell as part of the cellular process(es) capable of duplicating the the genetic element.

A “TATA box”, or “TATA element” refers to a nucleotide sequence element, common in many promoters, which binds a general transcription factor and hence specifies the position where transcription is initiated. The TATA box is an important element for transcription of sequences whose expression is dependent on type III RNA polymerase III promoters. As the name implies, the TATA box typically comprises the nucleic acid sequence 5′-TATA-3′ (or variations thereof known in the art).

“Terminators” or “termination sequence” refers to those DNA sequences that cause transcription of a nucleic acid sequence to cease. A termination sequence may be recognized intrinsically by the polymerase, or termination may require additional termination factors to be effective. Each of the three eukaryotic polymerases stops synthesizing RNA in response to different termination sequences. Eukaryotic RNA polymerases I and II generally require factors in addition to nucleic acid sequence elements to effect transcription termination. Eukaryotic RNA polymerase III however, recognizes termination sequences accurately and efficiently in the apparent absence of other factors. Simple clusters of four or more thymidine residues serve as terminators in most cases.

The term “vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

siRNA

RNA interference refers to the process of sequence specific post transcriptional gene silencing in animals mediated by short interfering RNAs (siRNA) (Fire et al., Nature, 391:806 (1998)). The corresponding process in plants is commonly referred to as post transcriptional gene silencing or RNA silencing and in fungi is also referred to as “quelling”. The process of post transcriptional gene silencing is thought to be an evolutionarily conserved cellular defense mechanism used to prevent the expression of foreign genes which is commonly shared by diverse flora and phyla (Fire et al., Trends Genet., 15:358 (1999)). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA.

The presence of long dsRNAs in cells stimulates the activity of a ribonuclease III enzyme referred to as “dicer.” Dicer is involved in the processing of the dsRNA into short pieces of dsRNA known as short interfering RNAs (siRNA) (Berstein et al., Nature, 409:363 (2001)). Short interfering RNAs derived from dicer activity are typically about 21-23 nucleotides in length and comprise about 19 base pair duplexes. The RNAi response also features an endonuclease complex containing a siRNA, commonly referred to as a “RNA-induced silencing complex” (RISC), which mediates cleavage of single stranded RNA having sequence homologous to the siRNA. Cleavage of the target RNA takes place in the middle of the region complementary to the guide sequence of the siRNA duplex (Elbashir et al., Genes Dev., 15:188 (2001)).

Chemically synthesizing nucleic acid molecules with modifications (base, sugar and/or phosphate) can prevent their degradation by serum ribonucleases, which can increase their potency, see e.g., Eckstein et al., International Publication No. WO 92/07065; Perrault et al., Nature 344:565-568 (1990); Pieken et al., Science 253:314-371 (1991); Usman and Cedergren, Trends in Biochem. Sci. 17:334 (1992); Usman et al., International Publication No. WO 93/15187; and Rossi et al., International Publication No. WO 91/03162; Sproat, U.S. Pat. No. 5,334,711; and Gold et al., U.S. Pat. No. 6,300,074. All of the above references describe various chemical modifications that can be made to the base, phosphate and/or sugar moieties of nucleic acid molecules, including modifications to shorten oligonucleotide synthesis times and reduce chemical requirements.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-O-allyl, 2′-H, nucleotide base modifications (for a review see Usman and Cedergren, TIBS. 17:34 (1992); Usman et al., Nucleic Acids Symp. Ser. 31:163 (1994); Burgin et al., Biochemistry, 35:14090 (1996)). Sugar modification of nucleic acid molecules have been described in the art (see, e.g., the references cited in the preceding paragraph and Beigelman et al., J. Biol. Chem., 270: 25702 (1995); Beigelman et al., International PCT publication No. WO 97/26270; Beigelman et al., U.S. Pat. No. 5,716,824; and Usman et al., U.S. Pat. No. 5,627,053. Such publications describe general methods and strategies to determine the location of incorporation of sugar, base and/or phosphate modifications and the like into nucleic acid molecules without modulating catalysis. In view of such teachings, similar modifications can be used as described herein to modify the siRNA nucleic acid molecules of the invention so long as the ability of the siRNA to promote RNAi is cells is not significantly inhibited (that is, by more than 25% and, in reverse order of preference, by 20%, 15%, 10%, or less). Other modifications to siRNA that enhance activity are discussed in McSwiggert, U.S. Patent Application 20030190635, published Oct. 9, 2003.

General Recombinant Methods

The expression cassettes and vectors of the present invention may be constructed utilizing standard techniques that are well known to those of ordinary skill in the art, such as those taught in Sambrook et al., Molecular Cloning—A Laboratory Manual (2nd ed.) Vol. 1-3, Cold Spring Harbor Laboratory, Cold Spring Harbor Press, N.Y., 1989 (hereafter “Sambrook”) (1989); Gelvin, S. B., Schilperoort, R. A., Varma, D. P. S., eds. Plant Molecular Biology Manual (1990)).

In preparing the expression cassettes of the present invention, the various DNA sequences may normally be inserted or substituted into a bacterial plasmid. Any convenient plasmid may be employed, which will be characterized by having a bacterial replication system, a marker which allows for selection of transformed bacteria and generally one or more unique, conveniently located restriction sites. These plasmids, referred to as vectors, may include such vectors as pACYC184, pACYC177, pBR322, pUC9, or pBluescript II (KS or SK), the particular plasmid being chosen based on the nature of the markers, the availability of convenient restriction sites, copy number, and the like. Thus, the sequence may be inserted into the vector at an appropriate restriction site(s), the resulting plasmid used to transform the E. coli host, the E. coli grown in an appropriate nutrient medium, and the cells harvested and lysed and the plasmid recovered. One then defines a strategy that allows for the stepwise combination of the different fragments.

It will be appreciated that the practice of the present invention involves generating alterations in nucleic acid sequences, which may be accomplished utilizing any of the methods known to one skilled in the art, including site-specific mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Berger and Kimmel, Guide to Molecular Cloning Techniques, Methods in Enzymology, Volume 152 Academic Press, Inc., San Diego, Calif. (Berger); Sambrook; and Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Pirrung et al., U.S. Pat. No. 5,143,854; and Fodor et al., Science, 251:767-77 (1991). Using these techniques, it is possible to insert or delete, at will, a polynucleotide of any length into an expression cassette of the present invention.

The practice of the present invention also involves chemical synthesis of linear oligonucleotides which may be carried out utilizing techniques well known in the art. The synthesis method selected will depend on various factors including the length of the desired oligonucleotide and such choice is within the skill of the ordinary artisan. Oligonucleotides are typically synthesized chemically according to the solid phase phosphoramidite triester method described by Beaucage and Caruthers, Tetrahedron Letts., 22(20):1859-1862 (1981), e.g., using an automated synthesizer, as described in Needham-VanDevanter et al., Nucleic Acids Res., 12:6159-6168 (1984). Oligonucleotides can also be custom made and ordered from a variety of commercial sources known to persons of skill in the art.

Synthetic linear oligonucleotides may be purified by polyacrylamide gel electrophoresis, or by any of a number of chromatographic methods, including gel chromatography and high pressure liquid chromatography. The sequence of the synthetic oligonucleotides can be verified using the chemical degradation method of Maxam and Gilbert. If modified bases are incorporated into the oligonucleotide, and particularly if modified phosphodiester linkages are used, then the synthetic procedures are altered as needed according to known procedures. In this regard, Uhlmann, et al., Chemical Reviews, 90:543-584 (1990) provide references and outline procedures for making oligonucleotides with modified bases and modified phosphodiester linkages.

The second strand of the coding nucleic acid of the invention typically is synthesized enzymatically. Enzymatic methods for DNA oligonucleotide synthesis frequently employ T7, T4, or Taq DNA polymerase or E. coli DNA polymerase I (holoenzyme or Klenow fragment) as described in, e.g., Sambrook. Enzymatic methods for RNA oligonucleotide synthesis frequently employ SP6, T3, or T7 RNA polymerase as described in Sambrook. Reverse transcriptase can also be used to synthesize DNA from RNA or DNA templates.

Linear oligonucleotides may also be prepared by polymerase chain reaction (PCR) techniques as described, for example, by Saiki et al., Science, 239:487 (1988). In vitro amplification techniques suitable for amplifying nucleotide sequences are also well known in the art. Examples of such techniques including the polymerase chain reaction (PCR), the ligase chain reaction (LCR), Qβ-replicase amplification and other RNA polymerase mediated techniques (e.g., NASBA) are found in Berger, Sambrook, and Ausubel, as well as Mullis et al., (1987) U.S. Pat. No. 4,683,202; PCR Protocols A Guide to Methods and Applications (Innis et al., eds) Academic Press Inc., San Diego, Calif. (1990) (Innis); Arnheim & Levinson (Oct. 1, 1990) C&EN 36-47; The Journal Of NIH Research, 3:81-94 (1991); Kwoh et al., Proc. Natl. Acad. Sci. USA, 86:1173 (1989); Guatelli et al., Proc. Natl. Acad. Sci. USA, 87:1874 (1990); Lomell et al., J. Clin. Chem, 35:1826 (1989); Landegren et al., Science, 241:1077-1080 (1988); Van Brunt, Biotechnology, 8:291-294 (1990); Wu and Wallace, Gene, 4:560 (1989); Barringer et al., Gene, 89:117 (1990), and Sooknanan and Malek, Biotechnology, 13:563-564 (1995). Improved methods of cloning in vitro amplified nucleic acids are described in Wallace et al., U.S. Pat. No. 5,426,039.

Vectors

siRNA molecules of the instant invention can be expressed within cells from eukaryotic promoters (e.g., Izant and Weintraub, Science, 229:345 (1985); McGarry and Lindquist, Proc. Natl. Acad. Sci., USA 83:399 (1986); Scanlon et al., Proc. Natl. Acad. Sci. USA, 88:10591-5 (1991); Sarver et al., Science, 247:1222-1225 (1990); Thompson et al., Nucleic Acids Res., 23:2259 (1995). Those skilled in the art will recognize that any nucleic acid can be expressed in eukaryotic cells from the appropriate DNA/RNA vector. The activity of such nucleic acids can be augmented by their release from the primary transcript by a enzymatic nucleic acid (e.g., Draper et al., PCT WO 93/23569, and Sullivan et al., PCT WO 94/02595; Ohkawa et al., Nucleic Acids Symp. Ser., 27:15-6 (1992); Taira et al., Nucleic Acids Res., 19:5125-30 (1991); Chowrira et al., J. Biol. Chem., 269:25856 (1994).

In another aspect of the invention, RNA molecules of the present invention can be expressed from transcription units (see, for example, Couture et al., TIG., 12:510 (1996)) inserted into DNA or RNA vectors. The recombinant vectors can be DNA plasmids or viral vectors. siRNA expressing viral vectors can be constructed based on, but not limited to, adeno-associated virus, retrovirus, adenovirus, or alphavirus. In another embodiment, pol III based constructs are used to express nucleic acid molecules of the invention (see for example Thompson, U.S. Pat. Nos. 5,902,880 and 6,146,886).

Recombinant vectors capable of expressing the siRNA molecules can be delivered as described herein and persist in target cells. Alternatively, viral vectors can be used that provide for transient expression of nucleic acid molecules. Such vectors can be administered additional times as necessary or desired. Once expressed, the siRNA molecule interacts with the target mRNA and generates an RNAi response.

In one aspect, the invention features an expression vector comprising a nucleic acid sequence encoding at least one siRNA molecule of the instant invention. The expression vector can encode one or both strands of a siRNA duplex, or a single self complementary strand that self hybridizes into a siRNA duplex. The nucleic acid sequences encoding the siRNA molecules of the instant invention can be operably linked in a manner that allows expression of the siRNA molecule (see for example Paul et al., 2002, Nature Biotechnology, 19:505 (2002); Miyagishi and Taira, Nature Biotechnology, 19:497 (2002); and Lee et al., Nature Biotechnology, 19:500 (2002).

In another aspect, the invention features an expression vector comprising: a) a transcription initiation region (e.g., eukaryotic pol I, II or III initiation region); b) a transcription termination region (e.g., eukaryotic pol I, II or III termination region); and c) a nucleic acid sequence encoding at least one of the siRNA molecules of the instant invention; wherein said sequence is operably linked to said initiation region and said termination region, in a manner that allows expression and/or delivery of the siRNA molecule.

Transcription of the siRNA molecule sequences can be driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87:6743-7 (1990); Gao and Huang 1993, Nucleic Acids Res., 21, 2867-72; Lieber et al., Methods Enzymol., 217:47-66 (1993); Zhou et al., Mol. Cell. Biol., 10, 4529-37 (1990)). Several investigators have demonstrated that nucleic acid molecules expressed from such promoters can function in mammalian cells (e.g. Kashani-Sabet et al., Antisense Res. Dev., 2:3-15 (1992); Ojwang et al., Proc. Natl. Acad. Sci. USA, 89: 10802-6 (1992); Lisziewicz et al., Proc. Natl. Acad. Sci. U.S.A, 90:8000-4 (1993); Thompson et al., Nucleic Acids Res., 23:2259 (1995); Sullenger & Cech, Science, 262:1566 (1993)). More specifically, transcription units such as the ones derived from genes encoding U6 small nuclear (snRNA), transfer RNA (tRNA) and adenovirus VA RNA are useful in generating high concentrations of desired RNA molecules such as siRNA in cells (e.g., Thompson et al., supra; Noonberg et al., Nucleic Acid Res., 22:2830 (1994); Noonberg et al., U.S. Pat. No. 5,624,803. The siRNA transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors).

In some embodiments of the present invention, vectors include, but are not limited to, chromosomal, nonchromosomal and synthetic DNA sequences (e.g., derivatives of viral DNA such as vaccinia, adenovirus, fowl pox virus, and pseudorabies). It is contemplated that any vector may be used as long as it is expressed and viable in the host; these criteria are sufficient for transient transfection. For stable transfection, the vector is also replicable in the host.

Large numbers of suitable vectors are known to those of skill in the art, and are commercially available. In some preferred embodiments of the present invention, mammalian expression vectors comprise an origin of replication, suitable promoters and enhancers, and also any necessary ribosome binding sites, polyadenylation sites, splice donor and acceptor sites, transcriptional termination sequences, and 5′ flanking non-transcribed sequences. In other embodiments, DNA sequences derived from the SV40 splice, and polyadenylation sites may be used to provide the required non-transcribed genetic elements.

In certain embodiments of the present invention, a gene sequence in the expression vector which is not part of an expression cassette encoding siRNA is operatively linked to an appropriate expression control sequence(s) (promoter) to direct mRNA synthesis. Promoters useful in the present invention include, but are not limited to, the cytomegalovirus (CMV) immediate early, herpes simplex virus (HSV) thymidine kinase, and mouse metallothionein-I promoters and other promoters known to control expression of gene in mammalian cells or their viruses. In other embodiments of the present invention, recombinant expression vectors include origins of replication and selectable markers permitting transformation of the host cell (e.g., dihydrofolate reductase or neomycin resistance for eukaryotic cell culture).

In some embodiments of the present invention, transcription of the DNA encoding a gene is increased by inserting an enhancer sequence into the vector. Enhancers are cis-acting elements of DNA, usually about from 10 to 300 bp that act on a promoter to increase its transcription. Enhancers useful in the present invention include, but are not limited to, a cytomegalovirus early promoter enhancer, the polyoma enhancer on the late side of the replication origin, and adenovirus enhancers.

In other embodiments, the expression vector also contains a ribosome binding site for translation initiation and a transcription terminator. In still other embodiments of the present invention, the vector may also include appropriate sequences for amplifying expression.

Exemplary vectors include, but are not limited to, the following eukaryotic vectors: pWLNEO, pSV2CAT, pOG44, PXT1, pSG (Stratagene) pSVK3, pBPV, pMSG, pSVL (Pharmacia). Particularly preferred plasmids are the Adenovirus vector (AAV; pCWRSV, Chatterjee et al. Science 258: 1485 (1992)), and pTZ18U (BioRad, Hercules, Calif., USA).

Administration of Nucleic Acids and Vectors

A large number of delivery methods for nucleic acids and for vectors are well known to those of skill in the art. Methods of non-viral delivery of nucleic acids include lipofection, microinjection, biolistics, virosomes, liposomes, immunoliposomes, polycation or lipid:nucleic acid conjugates, naked RNA, artificial virions, and agent-enhanced uptake of DNA.

In one method of administration, nucleic acids of the invention are introduced directly into superficial fat deposits by a jet of compressed gas or the like. Methods for introducing polynucleotides into body tissues such as the skin and muscle are well known and are taught, for example, in U.S. Pat. No. 5,830,877 and International Publication Nos. WO 99/52483 and 94/21797. The nucleic acid may be lyophilized and may be complexed, for example, with polysaccharides to form a particle of appropriate size and mass for acceleration into tissue. Conveniently, the nucleic acid can be placed on a gold bead or other particle which provides suitable mass or other characteristics. Use of gold beads to carry nucleic acids into body tissues to induce an immune response is taught in, for example, U.S. Pat. Nos. 4,945,050 and 6,194,389. Devices for accelerating particles (such as gold particles bearing nucleic acids) into body tissues using compressed gases are described in, for example, U.S. Pat. Nos. 6,592,545, 6,475,181, and 6,328,714.

In certain embodiments, the invention provides composition including dsRNA or dsRNA-encoding plasmids that are encapsulated or otherwise associated with liposomes. For example, dsRNA moieties or dsRNA-encoding plasmids can be condensed with a polycationic condensing agent, suspended in a low-ionic strength aqueous medium, and cationic liposomes formed of a cationic vesicle-forming lipid. The ratio of liposome lipids to plasmid can be adjusted to achieve maximum transfection. That ratio, in nmole liposome lipid/g plasmid, will often be greater than 5 but less than 25, and preferably greater than 8 but less than 18, and more preferably greater than 10 but less than 15 and most preferably between 12-14.

Liposomes are lipid vesicles having an outer lipid shell, typically formed on one or more lipid bilayers, encapsulating an aqueous interior. In a preferred embodiment, the liposomes are cationic liposomes composed of between about 20-80 mole percent of a cationic vesicle-forming lipid, with the remainder neutral vesicle-forming lipids and/or other components. As used herein, “vesicle-forming lipid” refers to any amphipathic lipid having hydrophobic and polar head group moieties and which by itself can form spontaneously into bilayer vesicles in water, as exemplified by phospholipids. A preferred vesicle-forming lipid is a diacyl-chain lipid, such as a phospholipid, whose acyl chains are typically between about 14-22 carbon atoms in length, and have varying degrees of unsaturation.

A cationic vesicle-forming lipid is one whose polar head group with a net positive charge, at the operational pH, e.g., pH 4-9. Typical examples include phospholipids, such as phosphatidylethanolamine, whose polar head groups are derivatized with a positive moiety, e.g., lysine. Also included in this class are the glycolipids, such as cerebrosides and gangliosides having a cationic polar head-group.

Use of liposomes to deliver therapeutic agents is well known in the art. Typically, the surface of the liposomes is modified by use poly(ethylene glycol) to slow clear from the circulation. Such liposomes resist opsonization and elimination by the mononuclear phagocytic system (MPS or RES), thereby enabling longer blood circulation times and enhanced tissue exposure for the encapsulated drug (Lasic et al. Chem. Rev. 95:2601-2627 (1995); Ishiwata et al., Chem. Pharm. Bull. 43:1005-1011 (1995)). The long-circulating liposomes enhance the pharmacokinetics and pharmacodynamics of RNA, particularly compared to conventional cationic liposomes which are known to accumulate in tissues of the MPS (Liu et al., J. Biol. Chem. 42:24864-24870 (1995); Choi et al., International PCT Publication No. WO 96/10391; Ansell et al., International PCT Publication No. WO 96/10390; Holland et al., International PCT Publication No. WO 96/10392). Long-circulating liposomes are also likely to protect nucleic acids from nuclease degradation to a greater extent compared to cationic liposomes, based on their ability to avoid accumulation in metabolically aggressive MPS tissues such as the liver and spleen.

The preparation of lipid:nucleic acid complexes, including targeted liposomes such as immunolipid complexes, is well known to those of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); and U.S. Pat. Nos. 4,186,183, 4,217,344, 4,235,871, 4,261,975, 4,485,054, 4,501,728, 4,774,085, 4,837,028, and 4,946,787).

Lipofection is described in, e.g., U.S. Pat. No. 5,049,386, U.S. Pat. No. 4,946,787; and U.S. Pat. No. 4,897,355 and lipofection reagents are sold commercially (e.g., Transfectam and Lipofectin). Cationic and neutral lipids that are suitable for efficient receptor-recognition lipofection of polynucleotides include those of Felgner, WO 91/17424, WO 91/16024. Delivery can be to cells or to target tissues.

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); and PCT/US94/05700).

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994)). Construction of recombinant AAV vectors are described in a number of publications, including U.S. Pat. No. 5,173,414; Tratschin et al., Mol. Cell. Biol. 5:3251-3260 (1985); Tratschin et al., Mol. Cell. Biol. 4:2072-2081 (1984); Hermonat & Muzyczka, Proc. Natl. Acad. Sci. U.S.A. 81:6466-6470 (1984); and Samulski et al., J. Virol. 63:03822-3828 (1989).

In particular, at least six viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent.
pLASN and MFG-S are examples are retroviral vectors that have been used in clinical trials (Dunbar et al., Blood 85:3048-305 (1995); Kohn et al., Nat. Med. 1:1017-102 (1995); Malech et al., Proc. Natl. Acad. Sci. U.S.A. 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors (Ellem et al., Immunol Immunother. 44(1):10-20 (1997); Dranoff et al., Hum. Gene Ther. 1:111-2 (1997)).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system (Wagner et al., Lancet 351:9117 1702-3 (1998), Kearns et al., Gene Ther. 9:748-55 (1996)).

Replication-deficient recombinant adenoviral vectors (Ad) are predominantly used transient expression gene therapy, because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 241:5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., Proc. Natl. Acad. Sci. U.S.A. 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Vectors (e.g., retroviruses, adenoviruses, liposomes, etc.) containing therapeutic nucleic acids can be also administered directly to the organism for transduction of cells in vivo. Alternatively, naked RNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Compositions for Administration

As indicated in the preceding section, the nucleic acid molecules of the invention and formulations thereof can be administered by a number of methods known in the art. In some embodiments, the nucleic acids are administered as naked RNA or plasmids or on carrier particles. If desired, the nucleic acids can be in polymers, which are preferably biodegradable within the time period over which release of the RNAi construct is desired or relatively soon thereafter, generally in the range of one year, more typically a few months, even more typically a few days to a few weeks. “Biodegradation” can refer to either a breakup of the microparticle, that is, dissociation of the polymers forming the microparticles and/or of the polymers themselves. This can occur as a result of change in pH from the carrier in which the particles are administered to the pH at the site of release, as in the case of the diketopiperazines, hydrolysis, as in the case of poly(hydroxy acids), by diffusion of an ion such as calcium out of the microparticle, as in the case of microparticles formed by ionic bonding of a polymer such as alginate, and by enzymatic action, as in the case of many of the polysaccharides and proteins. In some cases, linear release may be most useful, although in others a pulse release or “bulk release” may provide more effective results.

Formation of polymer matrices is well known in the art. Such methods as solvent evaporation, spray drying, solvent extraction and other methods known to those skilled in the art. Methods developed for making microspheres for drug delivery are described in the literature, for example, in Mathiowitz and Langer, J. Controlled Release 5,13-22 (1987); Mathiowitz, et al., Reactive Polymers 6, 275-283 (1987); and Mathiowitz, et al., J. Appl. Polymer Sci. 35, 755-774 (1988). The selection of the method depends on the polymer selection, the size, external morphology, and crystallinity that is desired, as described, for example, by Mathiowitz, et al., Scanning Microscopy 4,329-340 (1990); Mathiowitz, et al., J. Appl. Polymer Sci. 45, 125-134 (1992); and Benita, et al., J. Pharm. Sci. 73, 1721-1724 (1984). Further information about polymer formulations useful in delivering nucleic acids of the invention can be found in, e.g., U.S. Patent Application 20030157030.

In other embodiments, the compositions can be administered orally, parenterally, by inhalation or spray, or rectally in dosage unit formulations containing conventional pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like.

The invention provides formulations comprising a nucleic acid molecule of the invention and a pharmaceutically acceptable carrier. One or more nucleic acid molecules of the invention can be present in association with one or more pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palaetable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular subject depends upon a variety of factors including the activity of the specific compound employed, the age, body weight, general health, sex, diet, time of administration, route of administration, and rate of excretion, drug combination and the severity of the disorder undergoing therapy.

The nucleic acid molecules of the present invention may also be administered to a subject in combination with other therapeutic compounds to increase the overall therapeutic effect. The use of multiple compounds to treat an indication may increase the beneficial effects while reducing the presence of side effects.

### EXAMPLES

### Example 1

This Example sets forth materials and methods used in studies reported herein.

Cell Culture and Antibodies

The mouse 3T3-L1 preadipocyte cell line was obtained from American Tissue Culture Collection (ATCC). The cells were maintained in DMEM medium with 10% FBS, in a 37° C. incubator with 10% CO₂.

Rabbit antibodies against C/EBPβ, C/EBPα, PPARγ and IGF-1 receptor β subunit were purchased from Santa Cruz Biotechnologies, Inc., and used at 1:1000 (0.2 μg/ml) for Western blotting; those to ERK and Akt were from Cell Signaling Technology; mouse monoclonal antibody to phospho-tyrosine (PY20), insulin receptor β subunit and IRS-1 antibodies were from BD Biosciences. Rabbit anti-galectin-12 antibody was generated by immunizing rabbits with inclusion bodies of the C-terminal CRD of galectin-12 produced with the expression vector pET-14b (Novagen) and the E. coli strain BL21-SI (Life Technologies). For affinity purification of the antibody, the C-terminal CRD of galectin-12 was expressed as a fusion protein to the bacterial NusA protein with the vector pET-43.1 (Novagen). The fusion protein was purified with Talon affinity beads (Clontech) and conjugated to Sepharose 4B. These Sepharose affinity beads were then used to purify antibodies specific for galectin-12 from the antisera by affinity column chromatography.

RNA Interference with Small Interfering RNA (siRNA)

21-nucleotide siRNA duplexes with 3′UU overhangs on each strand targeting the American firefly luciferase, mouse galectin-3 and galectin-12 mRNAs were synthesized with the Silencer siRNA Construction Kit (Ambion). One control siRNA, siGL2, targets the sequence 5′-AACGUACGCGGAAUACUUCGA-3′ (SEQ ID NO:63) in the mRNA of the American firefly (Photinus pyralis) luciferase (Elbashir, S. M. et al. Nature, 411:494-498 (2001)). The other control siRNA, si3, targets the sequence 5′-AAACAGGAUUG UUCUAGAUUU-3′ (SEQ ID NO:64) in mouse galectin-3 mRNA. The three galectin-12 siRNAs (si12.1, si12.2 and si12.3) target the following distinct sequences in mouse galectin-12 mRNA: 5′-AAUUCCUGAACAUCAAUCCAU-3′ (SEQ ID NO:65), 5′-AACAUCAAU CCAUUUGUGGAG-3′ (SEQ ID NO:66), and 5′ AAUCUGGUGACAUCUUGGUAA-3′ (SEQ ID NO:67), respectively.

The transfection of 3T3-L1 cells with siRNA was performed using Oligofectamine (Invitrogen, San Diego, Calif.) (Elbashir, S. M. et al. Methods, 26:199-213 (2002)). Briefly, one day prior to transfection, approximately 2.5×10⁴ cells were seeded in wells of 6-well plates so that they would be ˜50% confluent the following day. The siRNA stocks were made by diluting siRNA duplexes into annealing buffer (10 mM Tris-HCl, 1 mM EDTA, 0.1 M NaCl, pH8) to 1 mM. Cells on each well were transfected with 10 μl Oligofectamine and 10 μl of 1 μM siRNA (final concentration 5 nM). One day after transfection, transfection mixture was removed and 4 ml fresh culture medium was added. Cells were induced to differentiate 3 days after transfection as described below.

Induction of Adipocyte Differentiation

To induce differentiation, confluent cells were exposed to a prodifferentive regimen consisting of 1 μM dexamethasone, 0.2 mM isobutylmethylxanthine, and 10 μg/ml insulin (Sigma) in culture medium for two days. The cells were subsequently cultured in medium with insulin only. Cell cycle distribution during adipocyte differentiation was determined by flow cytometry of propidium iodide-stained cells fixed with ethanol, as described previously (Yang, R. Y. et al. J Biol. Chem. 276:20252-20260 (2001)).

Lipid droplets in adipocytes were stained with the lipophilic dye Oil Red O (Catalano, R. A. et al. Stain Technol. 50:297-299 (1975); Koopman, R. et al. Histochem Cell Biol. 116:63-68 (2001)). Cells were fixed for 1 h in 3.7% formaldehyde, rinsed in 60% isopropanol and stained with 1.8 mg/ml Oil Red O (Sigma) in 60% isopropanol/0.4% dextrin for 15 min. Excessive Oil Red O stain was then washed away with three changes of water (30 s each), with the last one containing 1:1000 SYBR Green I (Molecular Probes) to stain the nuclei. Stained cells were visualized on a Zeiss confocal microscope equipped with argon 488 nm, helium-neon 543 nm and 633 nm lasers, using FITC filter set and Rhodamine filter set for SYBR Green I and Oil Red O, respectively. Images were acquired and analyzed with the LSM 510 software (Zeiss). Quantification of Oil Red O staining was achieved by extracting lipid-associated dye with 100% isopropanol for 15 min before spectrophotometry to measure the absorbance at 510 nm (Janderova et al., 2003).

Analysis of Gene Expression

RNA extraction and RT-PCR were performed as described (Yang, R. Y. et al. J Biol. Chem. 276:20252-20260 (2001)). For mouse galectin-12, primers 5′-CCTGCTCAC GTGCTCTTCCTCG-3′(SEQ ID NO:68) and 5′-TTGGAGCCCTTCTTAGCAGTGG-3′ (SEQ ID NO:69) were used. Primers 5′-TGAAGGTCGGTGTGAACGGATTTGGC-3′ (SEQ ID NO:70) and 5′-CATGTAGGCCATGAGGTCCACCAC-3′(SEQ ID NO:71) were used for mouse GAPDH as control. For Western blotting, cells were lyzed in SDS sample buffer, boil for 5 min and the lysates were resolved on SDS-polyacrylamide gels, transferred to Immobilon-P (Millipore) membrane, and probed with indicated antibodies, as described (Yang, R. Y. et al. J Biol. Chem. 276:20252-20260 (2001)).

Detection of Receptor Tyrosine Phosphorylation

Cells were lysed in NP-40 lysis buffer ((Yang, R. Y. et al. J Biol. Chem. 276:20252-20260 (2001)) containing 1 mM sodium vanadate, and 200 μg of protein was incubated for 2 h with 2 μg of PY20 antibody and 20 ml of Protein G-Sepharose (Pharmacia) to precipitate tyrosine-phosphorylated proteins. After 3 washes with lysis buffer, the precipitated proteins were eluted by boiling 5 min in 10 ml SDS sample buffer, resolved by 10% SDS-PAGE and subjected to Western blotting with anti-IGF1 receptor antibody.

### Example 2

This Example sets forth results of studies performed in the course of the present invention.

Comparison of Human and Mouse Galectin-12 Genes

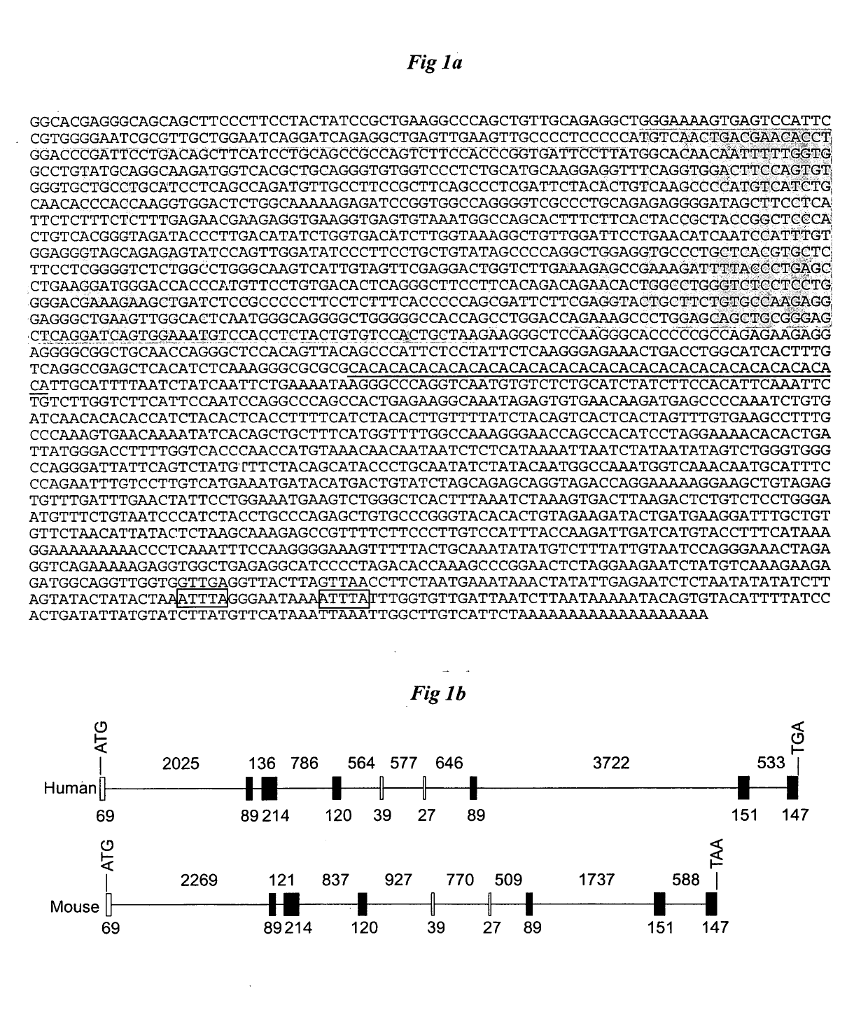

By BLAST search of the EST database with human galectin-12 cDNA (Yang, R. Y. et al. J Biol. Chem. 276:20252-20260 (2001)), we found a mouse EST sequence (Genbank Acc: AA184213) with the highest homology to the query. The EST clone was then obtained from Research Genetics and sequenced in full. The 2468-bp sequence contains a 945-bp major open reading frame (ORF) of 85% identity to that of human galectin-12. Like human galectin-12 cDNA, the start codon of the mouse ORF lies in a suboptimal context for translation initiation, and the 3′-untranslated region (3′-UTR) of the cDNA contains 2 AT-rich elements. In addition, there are 25 consecutive CA repeats in the 3′UTR (FIG. 1a). BLAST search of the mouse genome with human galectin-12 cDNA revealed a genomic sequence on mouse chromosome 19 with highest homology. Alignment between the two sequences identified exon/intron boundaries in the genomic sequence, and assembly of the exon sequences gave rise to a sequence of 100% identity to the mouse cDNA identified above. Furthermore, comparison of human galectin-12 gene and this mouse genomic sequence revealed striking similarities in exon organization, with all corresponding exons are of the same lengths (FIG. 1b). We thus identified mouse galectin-12 gene and its coding sequence.

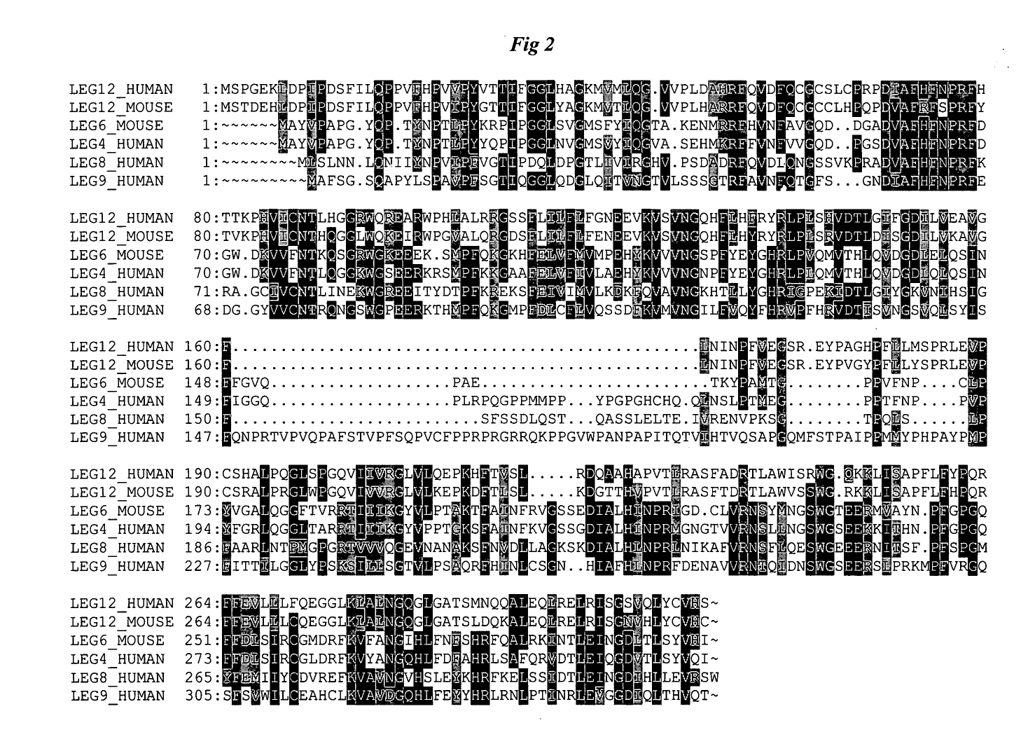

The mouse galectin-12 cDNA encodes a protein of 81% identity to the human galectin-12 polypeptide. The homology distributes evenly over the two sequences, with identities of 80%, 88% and 81% for the N-terminal CRDs, the linker regions, and the C-terminal CRDs, respectively. The N-terminal CRD of mouse galectin-12 is similar to those of other galectins, while the linker sequence, which is 88% identical to that of human galectin-12, does not share homology with any other galectins. Likewise, its C-terminal CRD, while highly similar to that of human galectin-12 (81% identity), is clearly distinct from other galectin CRDs (FIG. 2).

Expression of Galectin-12 in Preadipocytes and During Adipocyte Differentiation Correlates with the Cell Cycle Status

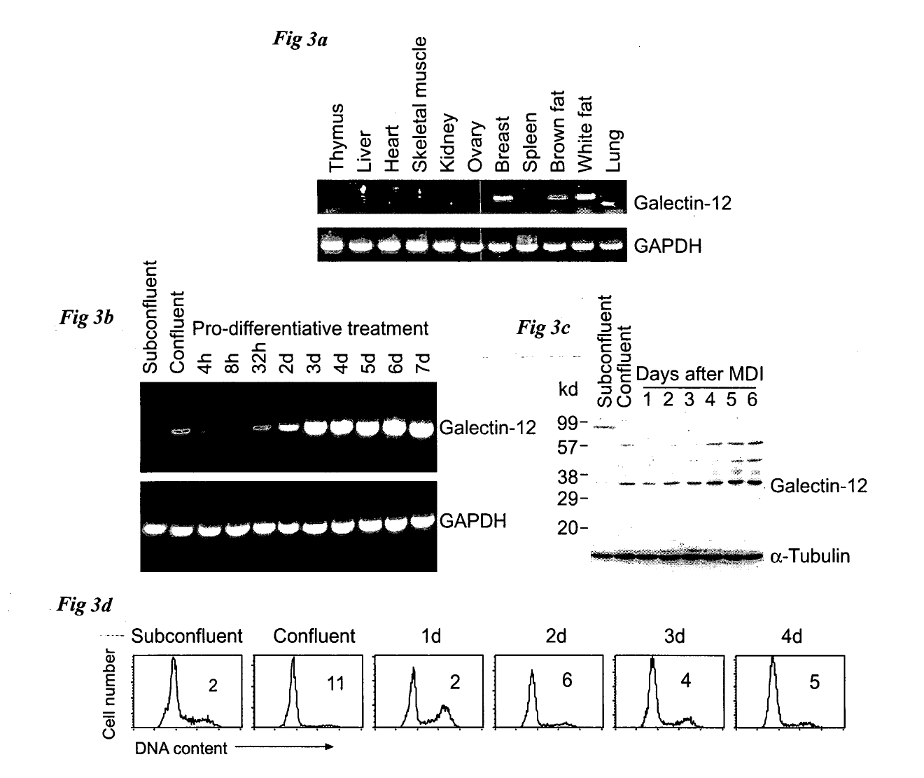

We determined the expression of galectin-12 in multiple mouse tissues by RT-PCR and found that like its human counterpart, mouse galectin-12 gene is also preferentially expressed in adipose tissue (FIG. 3a). Both brown and white fat express galectin-12 mRNA abundantly. The breast also shows high galectin-12 expression, while other tissues examined exhibit either low or no expression of this gene (FIG. 3a). The preadipocyte cell line 3T3-L1 is a continuous substrain of 3T3 (Swiss albino) developed through clonal isolation (Green, H. et al. Cell. 3:127-133 (1974)). These cells become competent to undergo a pre-adipose to adipose like conversion as they progress from a rapidly dividing to a confluent and contact inhibited state, and are frequently used in an in vitro model of adipocyte differentiation that closely recapitulates the in vivo process (Gregoire, F. M. et al. Physiol Rev. 78:783-809 (1998)). To determine the timing of galectin-12 expression in adipocyte differentiation, galectin-12 mRNA and protein were detected by RT-PCR and Western blotting, respectively, during the differentiation of 3T3-L1 cells. Consistent with our previous observation that galectin12 arrests the cell cycle at G1 phase, we found that its expression was low in rapidly dividing subconfluent 3T3-L1 cells, but significantly up-regulated when cells undergo growth arrest as the culture reached confluence (FIGS. 3b and c). Treatment of confluent cultures with adipogenic hormones, which induce transient mitotic clonal expansion during the first day (Tang, Q. Q. et al. Proc Natl Acad Sci USA. 100:44-49 (2003)), as indicated by the re-appearance of cells at the S and G2/M phase of the cell cycle (FIG. 3d), down-regulated galectin-12 expression in just a few hours (FIG. 3a). Galectin-12 mRNA and protein levels were again markedly elevated two days after adipogenic hormone stimulation (FIGS. 3b and c), when cells entered the terminal stage of differentiation and permanently exited the cell cycle (Reichert, M. et al. Oncogene, 18:459-466 (1999)) (FIG. 3c).

Galectin-12 is Required for Adipocyte Differentiation

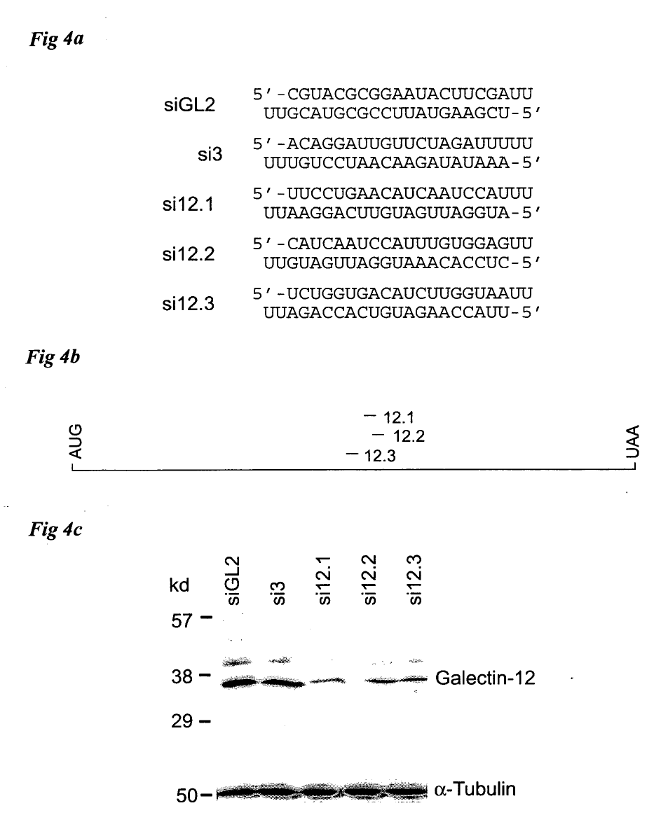

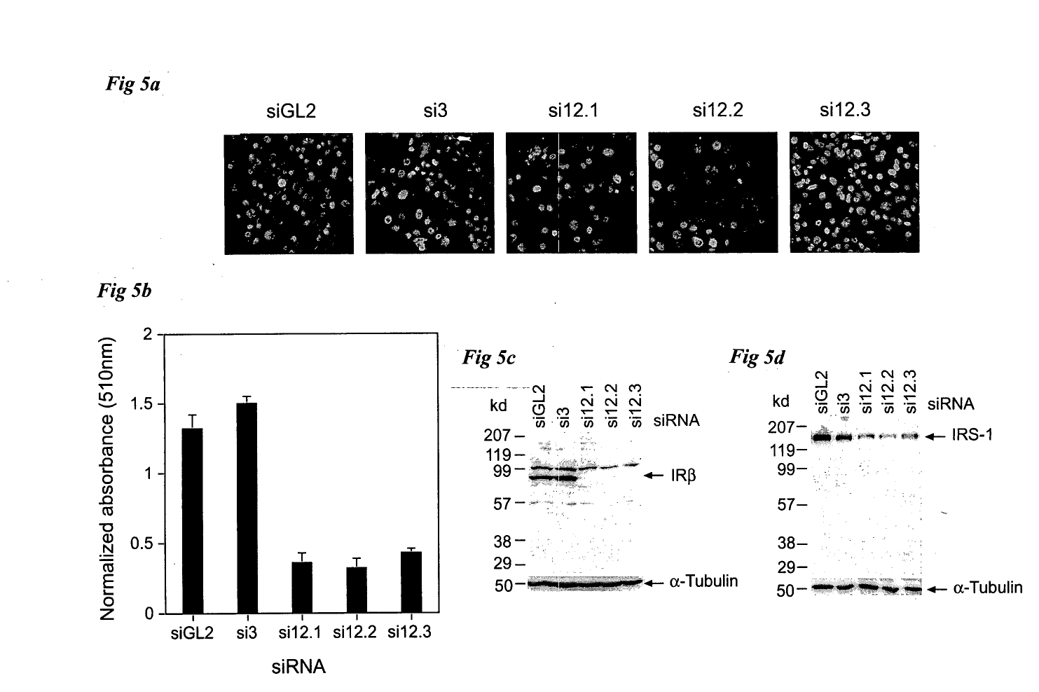

We employed RNA interference to study the role of galectin-12 in adipocyte differentiation, by knocking down the expression of endogenous galectin-12 with small interfering RNAs specific for galectin-12 mRNA (FIG. 4). It has been shown that siRNA with 2-nucleotide overhangs efficiently and specifically suppresses gene expression (Chi, J. T. et al. Proc Natl Acad Sci USA. 100:6343-6346 (2003); Elbashir, S. M. et al. Nature, 411:494-498 (2001); Semizarov, D. et al. Proc Natl Acad Sci USA. 100:6347-6352 (2003)). Two control siRNAs, for luciferase and galectin-3, respectively, and 3 siRNAs targeting different sequences in galectin-12 mRNA were used in our experiments (FIGS. 4a and b). When 3T3-L1 cells were treated with siRNAs before subjected to the pro-differentiative regimen, all 3 galectin-12 siRNAs significantly down-regulated galectin-12 expression, while two control siRNAs did not affect the expression (FIG. 4c). After stimulation with adipogenic hormones, control siRNA transfected cells accumulated large quantities of lipid droplets, a telltale sign of adipocyte differentiation. In contrast, cells transfected with galectin-12 siRNAs contained only small amounts of lipid droplets. (FIG. 5a). Quantitation of Oil Red O staining indicated that cells transfected with galectin-12 siRNAs accumulated only ⅓ as much triglyceride seen in control cells (FIG. 5b), indicating a major defect in adipogenesis. Aside from serving as a depot for triglyceride, another important function for adipose tissue is to regulate glucose metabolism in response to insulin stimulation. Insulin sensitivity is acquired during adipocyte differentiation as insulin receptor and its substrates are upregulated by C/EBPα (Wu, Z. et al. Mol Cell. 3:151-158 (1999)). Western blotting revealed high levels of insulin receptor expression in control cells 7 days after adipogenic treatments. In contrast, no insulin receptor expression was detectable after the same treatment of cells transfected with galectin-12 siRNAs (FIG. 5c). These cells also expressed less insulin receptor substrate-1 (IRS-1) than control cells (FIG. 5d).

Impaired Induction of Critical Adipogenic Transcription Factors in Galectin-12-Deficient Cells

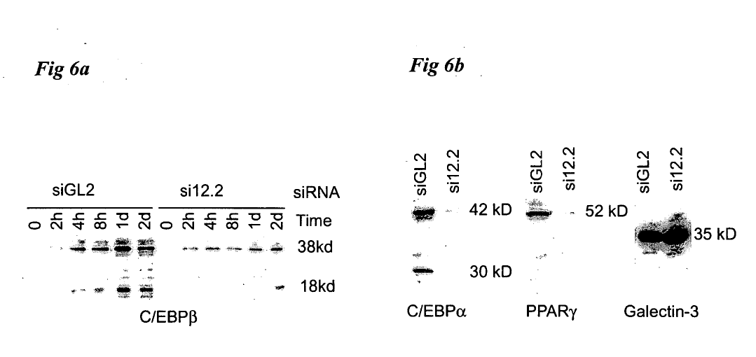

Based on their temporal sequence of expression, as well as gain- and loss-of-function studies both in vitro and in vivo, it is now well recognized that C/EBPβ, and to a less extent C/EBPδ, activate the two major adipogenic transcription factors C/EBPα and PPARγ (Rosen, E. D. et al. Genes Dev. 14:1293-1307 (2000)). PPARγ-activated gene expression is responsible for most of the mature adipocyte phenotype (Rosen, E. D. et al. Genes Dev. 16:22-26 (2002)), except insulin sensitivity, which is ascribed to the C/EBPα-induced expression of insulin receptor and IRS-1 genes (Wu, Z. et al. Mol Cell. 3:151-158 (1999)). Since adipogenic treatment fails to induce adipogenesis and insulin sensitivity in galectin-12-deficient cells (FIG. 5), we tested whether galectin-12 is required for the induction of these critical transcription factors. In control cells, adipogenic hormonal treatment induced C/EBPβ expression in 2 hours. C/EBPβ protein levels in these cells continued to rise and peaked after one day of treatment (FIG. 6a). Although C/EBPβ was also induced in cells transfected with galectin-12 siRNA, its expression levels remained low during two days of adipogenic hormone treatment of these cells (FIG. 6a). Accordingly, Western blotting with specific antibodies 5 days after adipogenic hormone stimulation revealed greatly reduced expression of C/EBPα and PPARγ, the two downstream targets for C/EBPβ, in galectin-12 siRNA transfected cells, with no changes in galectin-3 expression (FIG. 6b).

Activation of Protein Kinases Important for Adipogenesis in Response to Adipogenic Hormone Stimulation is Defective in Galectin-12 Knockdown Cells

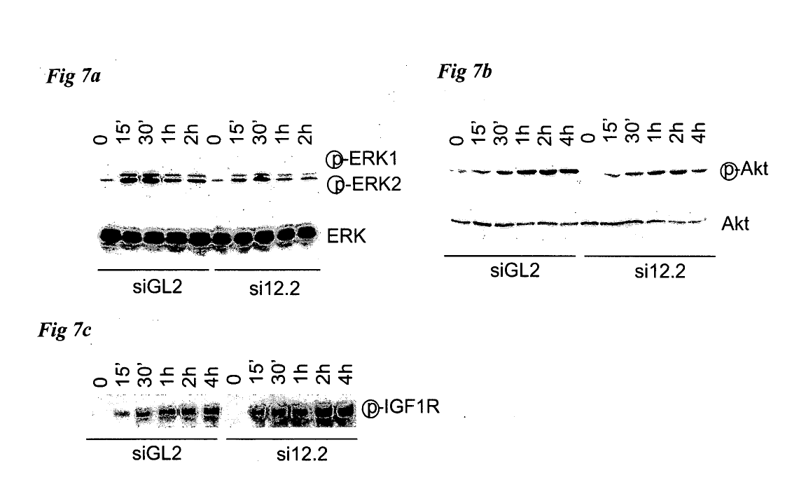

Signaling emanating from insulin/IGF receptors on preadipocytes activates the MAP kinase ERK and also Akt (Gagnon, A. et al. Diabetes, 48:691-698 (1999; Sorisky, A. Crit Rev Clin Lab Sci. 36:1-34 (1999)). Early activation of ERK is required for C/EBPβ and 6 induction and subsequent adipocyte differentiation (Belmonte, N. et al. Mol Endocrinol. 15:2037-2049 (2001)), although later activation of this kinase results in the phosphorylation and inactivation of PPARg and inhibits adipogenesis (Camp, H. S. et al. J Biol. Chem. 272:10811-10816 (1997); Hu, E. et al. Science, 274:2100-2103 (1996)). Akt/PKB is also known to promote adipogenesis (Magun, R. et al. Endocrinology, 137:3590-3593 (1996); Peng et al. Genes Dev. 17:1352-1365 (2003)). Since galectin-12 is required for the induction of C/EBPβ (FIG. 6), we set out to determine the effect of galectin-12 knockdown on the upstream signaling, and found that the phosphorylation of both ERK (FIG. 7a) and Akt (FIG. 7b) is down-regulated in galectin-12-deficient cells (FIG. 7). IGF-1 receptor and insulin receptor initiate more or less the same downstream signaling pathways after self-phosphorylation and recruitment of adaptor proteins Shc and IRS molecules, which channel the signal through the MAP kinase and PI3K/Akt pathways, respectively (Saltiel, A. R. et al. Nature, 414:799-806 (2001)). IGF-1 receptor is however the predominant receptor for insulin on preadipocytes, although it is taken over by insulin receptor as these cells differentiated into mature adipocytes (Sorisky, A. Crit Rev Clin Lab Sci. 36:1-34 (1999)). We therefore investigated whether self-phosphorylation of IGF-1 receptor is affected in galectin-12-deficient cells. Western blotting of PY20-precipitated proteins with an antibody to IGF-1 revealed that tyrosine phosphorylation of IGF-1 receptor is not reduced (FIG. 7c).

### Example 3

This Example sets forth a discussion of results set forth in the preceding Example.

Many important features are conserved in mouse and human galectin-12 mRNAs (FIG. 1b). The location of the translation initiation codon in a sequence context suboptimal for translation initiation again suggests that under normal conditions, mouse galectin-12 mRNA translation is tightly controlled at the level of translation initiation. The presence of AU-rich motifs in the 3′-UTR indicates another level of control on mRNA stability. Stringent regulation of expression is critical for genes of functions in cell growth and development, because their aberrant expression will have grave consequences. The significance of the presence of 25 consecutive CA repeats in the 3′-UTR or mouse galectin12 mRNA, which is not found in its human counterpart, is not known, but it may also have regulatory functions. These structural features are unique for galectin-12 mRNA and are absent from the transcripts of other galectins.

Galectin-12 shows clear differences from other galectins at the level of protein sequence (FIG. 2). While its N-terminal CRD is similar to those of other galectins, the C-terminal one exhibits significant divergence, suggesting that this CRD may be on its way to evolve into a domain of novel functions. In contrast, the N- and C-terminal CRDs of other 2-CRD galectins are very similar (FIG. 2).

Like its human counterpart, mouse galectin-12 is also preferentially expressed in adipose tissue (FIG. 3). White and brown adipose tissues are functionally opposite. While white adipose tissue (WAT) serves as an energy depot by storing it in the form of triglyceride, brown adipose tissue (BAT) dissipates energy in the form of heat through uncoupling of electron transfer by the protein UCP-1, which is expressed exclusively in BAT and not WAT (Rosen, E. D. et al. Genes Dev. 14:1293-1307 (2000)). The development of WAT and BAT, on the other hand, is under similar molecular control that critically involves proteins of the C/EBP family and PPARγ (Rosen, E. D. et al. Genes Dev. 14:1293-1307 (2000)). The expression of galectin-12 in both tissues (FIG. 3a) thus suggests a function in adipogenesis. This was again supported by its expression in the 3T3-L1 cell line of the adipocyte lineage and the changes in its expression during adipocyte differentiation (FIG. 3b).

Previous studies of galectin functions have frequently involved the use of purified recombinant proteins expressed in E. Coli, or over-expression by transfection of the target cell types. To what extent the function of the protein in question can be inferred from these studies is not certain because it's not known whether such levels of expression can be achieved in physiological or pathological conditions. Furthermore, galectin may function intracellularly, and effects observed by simply adding exogenous recombinant proteins to cell cultures may not be relevant. RNA interference, on the other hand, helps delineate protein functions by down-regulating the expression of endogenous protein, and is therefore a better alternative for functional studies. Unlike gene targeting by replacement or insertion through homologous recombination, RNA interference does not result in the expression of the target being completely eliminated (FIG. 4). While partial reduction of expression may not be enough to reveal the roles of some other genes whose function can be achieved even when expressed at low levels, our studies herein found partial reduction sufficient to determine the role of galectin-12, whose expression is normally under tight control. Thus, we thought even a slight reduction in expression would have significant consequences. Indeed, interference of galectin-12 expression with siRNAs markedly reduced adipogenesis (FIG. 5).

Our results show that galectin-12 is required for optimal signaling of hormonal stimulation to the induction of adipogenic factors important for adipocyte differentiation. This is the first galectin to be shown to modulate the differentiation of a key cell type of metabolism, by regulating the signaling pathway instrumental to energy storage and expense (Saltiel, A. R. et al. Nature, 414:799-806 (2001)). Our results clearly implicate galectin-12 in the establishment of a competent state in growth-arrested preadipocytes to respond to hormonal stimulation and undergo adipocyte differentiation.

The nature of galectin-12's involvement in insulin signaling is not known. It was previously reported that galectin-12 localizes in the cytoplasm in speckled pattern, as well as in the nucleus Hotta, K. et al. J Biol. Chem. 276:34089-34097 (2001)). While its presence in the nucleus, where the central cell cycle machinery is located, is consistent with its function in cell cycle regulation, cytoplasmic distribution is in agreement with an additional role for galectin-12 in the regulation of proximal signalling events. The fact that tyrosine phosphorylation of IGF-1 receptor is not reduced but activation of downstream kinases is defective (FIG. 4) suggests that galectin-12 acts downstream of IGF-1/insulin receptors and upstream of ERK and Akt. This probably occurs at the level of the adaptor proteins Shc and IRS, in particular their recruitment and phosphorylation by the receptors. Galectin-12 may directly modulate these signaling events through physical interactions with relevant components of the signaling pathway. In fact, galectins have been shown to interact with other proteins independent of binding to carbohydrates, although these interactions are often lactose-inhibitable and involve their CRDs (Seve, A. P. et al. Exp Cell Res. 213:191-197 (1994); Yang, R. Y. et al. Biochemistry, 37:4086-4092 (1998); Yang, R. Y. et al. Proc Natl Acad Sci USA. 93:6737-6742 (1996)).

Clear divergence of the C-terminal CRD of galectin-12 from traditional galectin CRDs (Yang, R. Y. et al. J Biol. Chem. 276:20252-20260 (2001)) makes it more likely that this CRD is not intended for binding to carbohydrate, but to ligands of other nature instead. Alternatively, due to galectin-12's growth suppression activity, cells may interpret low galectin-12 expression as oncogenic and respond by down-regulating growth factor signaling to counter its impact on cell proliferation. Failure to do so would lead to cellular transformation (Rao, D. S. et al. Cancer Cell. 3:471-482 (2003)). Our data indicate that the initial up-regulation of galectin-12, when cells undergo growth arrest, is required for the response of preadipocytes to adipogenic hormone stimulation. In view of its anti-proliferative activity (Yang, R. Y. et al. J Biol. Chem. 276:20252-20260 (2001)), transient down-regulation of galectin-12 during the first day of hormonal treatment (FIG. 1a and b) may be essential for mitotic clonal expansion to occur. Subsequent up-regulation of galectin-12 expression after clonal expansion coincides with C/EBPα expression and may be important for the establishment of insulin sensitivity in mature adipocytes. Consistent with this, agents that cause insulin resistance in adipocytes down-regulate galectin-12 expression (Fasshauer, M. et al. Eur J Endocrinol. 147:553-559 (2002)).

Our results suggest that inhibitors of galectin-12 activity or expression can suppress adipocyte differentiation, and may be useful for controlling obesity and associated diseases. These agents could provide other benefits as well, because suboptimal signaling through insulin/IGF receptors has been shown to extend the life span of animals (Bluher, M. et al. Science, 299:572-574 (2003); Holzenberger, M. et al. Nature, 421:182-187 (2003)).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A short interfering (si) RNA comprising a first and a second strand, each strand (a) being of equal length, (b) from 16 to 29 nucleotides in length, (c) hybridized to the other strand, and (d) having a 5′ end and a 3′ end, and wherein in which the 16 to 29 nucleotides of one strand are complementary to a corresponding length of galectin-12 mRNA (SEQ ID NO:1).

2. A siRNA of claim 1 in which at least 1 nucleotide, but not more than 4 nucleotides, at the 5′ end of a strand is deoxyribose nucleic acid.

3. A siRNA of claim 1 in which at least 1 nucleotide, but not more than 4 nucleotides, at the 3′ end of a strand is deoxyribose nucleic acid.

4. A siRNA of claim 1, further comprising at least 1 unpaired nucleotide at the 3′ end of each strand.

5. A siRNA of claim 4, in which at least one unpaired nucleotide at the 3′ end of at least one strand is a deoxyribose nucleic acid.

6. A siRNA of claim 1, wherein one of said two strands has a sequence selected from the group consisting of SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30.

7. A siRNA of claim 6, further comprising at least 1 unpaired nucleotide at the 3′ end of each strand.

8. A siRNA of claim 7, in which at least 1 nucleotide, but not more than 4 nucleotides, at a 5′ end of a strand is deoxyribose nucleic acid.

9. A siRNA of claim 4, wherein said first and second strands are selected from the group consisting of (a) SEQ ID NOS:3 and 4, (b) SEQ ID NOS:5 and 6, (c) SEQ ID NOS:7 and 8, (d) SEQ ID NOS:9 and 10, (e) SEQ ID NOS:11 and 12, (f) SEQ ID NOS:13 and 14, (g) SEQ ID NOS:15 and 16, (h) SEQ ID NOS:31 and 32, (i) SEQ ID NOS:33 and 34, (j) SEQ ID NOS:35 and 36, (k) SEQ ID NOS:37 and 38, (l) SEQ ID NOS:39 and 40, (m) SEQ ID NOS:41 and 42, (n) SEQ ID NOS:43 and 44, (o) SEQ ID NOS:45 and 46, (p) SEQ ID NOS:47 and 48, (q) SEQ ID NOS:49 and 50, (r) SEQ ID NOS:51 and 52, (s) SEQ ID NOS:53 and 54, (t) SEQ ID NOS:55 and 56, (u) SEQ ID NOS:57 and 58, (v) SEQ ID NOS:59 and 60, and (w) SEQ ID NOS:61 and 62.

10. A siRNA of claim 1, wherein said first and said second strands are linked.

11. A siRNA of claim 9, wherein said first and said second strands are linked.

12. A vector comprising a first promoter operably linked to a nucleic acid comprising a first segment that encodes at least a first strand of a short interfering (si) RNA from 16 to 29 nucleotides in length, which said strand has a 5′ end and a 3′ end, in which said 16 to 29 nucleotides of said first strand are complementary to a corresponding length of galectin-12 mRNA (SEQ ID NO:1).

13. A vector of claim 12, further comprising a second segment that encodes a RNA complementary to that encoded by said first segment.

14. A vector of claim 12, further comprising a second segment that encodes a RNA complementary to that encoded by said first segment and a linker between said first segment and said second segment.

15. A vector of claim 12, further comprising a second promoter positioned to permit transcription of RNA in a direction antiparallel to said first promoter and which, when transcribed antiparallel to said first promoter, results in transcription of a RNA complementary to said first strand.

16. A vector of claim 12 wherein said siRNA further comprises at least 1 unpaired nucleotide at the 3′ end of said first strand.

17. A vector of claim 12 wherein said first strand has a sequence selected from the group consisting of SEQ ID NO:9, SEQ ID NO:10, SEQ ID NO:11, SEQ ID NO:12, SEQ ID NO:13, SEQ ID NO:14, SEQ ID NO:21, SEQ ID NO:22, SEQ ID NO:23, SEQ ID NO:24, SEQ ID NO:25, and SEQ ID NO:26.

18. A use of a short, interfering (si) RNA from 16 to 29 nucleotides in length, said siRNA having a first strand and a second strand, each strand being (a) of equal length, (b) from 16 to 29 nucleotides in length, (c) hybridized to the other strand, and (d) having a 5′ end and a 3′ end, and wherein in which the 16 to 29 nucleotides of one strand are complementary to a corresponding length of galectin-12 mRNA (SEQ ID NO:1), or a vector encoding such a siRNA, for manufacture of a medicament to inhibit differentiation of pre-adipocytes.

19. A use of claim 18, wherein said siRNA further comprises at least 1 unpaired nucleotide at the 3′ end of each strand.

20. A use of claim 18, wherein said strands are selected from the group consisting of (a) SEQ ID NOS:3 and 4, (b) SEQ ID NOS:5 and 6, (c) SEQ ID NOS:7 and 8, (d) SEQ ID NOS:9 and 10, (e) SEQ ID NOS:11 and 12, (f) SEQ ID NOS:13 and 14, (g) SEQ ID NOS:15 and 16, (h) SEQ ID NOS:31 and 32, (i) SEQ ID NOS:33 and 34, (j) SEQ ID NOS:35 and 36, (k) SEQ ID NOS:37 and 38, (l) SEQ ID NOS:39 and 40, (m) SEQ ID NOS:41 and 42, (n) SEQ ID NOS:43 and 44, (o) SEQ ID NOS:45 and 46, (p) SEQ ID NOS:47 and 48, (q) SEQ ID NOS:49 and 50, (r) SEQ ID NOS:51 and 52, (s) SEQ ID NOS:53 and 54, (t) SEQ ID NOS:55 and 56, (u) SEQ ID NOS:57 and 58, (v) SEQ ID NOS:59 and 60, and (w) SEQ ID NOS:61 and 62.

21. A use of an inhibitor of galectin-12 activity for manufacture of a medicament to inhibit differentiation of pre-adipocytes.

22. A use of an inhibitor of galectin-12 activity for manufacture of a medicament to inhibit differentiation of leukocytes.

23. A composition comprising a short, interfering (si) RNA from 16 to 29 nucleotides in length, said siRNA having a first strand and a second strand, each strand being (a) of equal length, (b) from 16 to 29 nucleotides in length, (c) hybridized to the other strand, and (d) having a 5′ end and a 3′ end, and wherein in which the 16 to 29 nucleotides of one strand are complementary to a corresponding length of galectin-12 mRNA (SEQ ID NO:1), and a pharmaceutically acceptable carrier.

24. A composition of claim 23, wherein said siRNA further comprises at least 1 unpaired nucleotide at the 3′ end of each strand.

25. A composition of claim 23, in which at least one unpaired nucleotide at the 3′ end of at least one strand is a deoxyribose nucleic acid.

26. A composition of claim 23 in which at least 1 nucleotide, but not more than 4 nucleotides, at a 5′ end of a strand is deoxyribose nucleic acid.

27. A composition of claim 23 in which at least 1 nucleotide, but not more than 4 nucleotides, at a 3′ end of a strand is deoxyribose nucleic acid.

28. A composition of claim 23, wherein one of said two strands has a sequence selected from the group consisting of SEQ ID NO:9, SEQ ID NO:10, SEQ ID NO:11, SEQ ID NO:12, SEQ ID NO:13, SEQ ID NO:14, SEQ ID NO:21, SEQ ID NO:22, SEQ ID NO:23, SEQ ID NO:24, SEQ ID NO:25, and SEQ ID NO:26.

29. A composition of claim 23, wherein said strands are selected from the group consisting of (a) SEQ ID NOS:3 and 4, (b) SEQ ID NOS:5 and 6, (c) SEQ ID NOS:7 and 8, (d) SEQ ID NOS:9 and 10, (e) SEQ ID NOS:11 and 12, (f) SEQ ID NOS:13 and 14, (g) SEQ ID NOS:15 and 16, (h) SEQ ID NOS:31 and 32, (i) SEQ ID NOS:33 and 34, (j) SEQ ID NOS:35 and 36, (k) SEQ ID NOS:37 and 38, (l) SEQ ID NOS:39 and 40, (m) SEQ ID NOS:41 and 42, (n) SEQ ID NOS:43 and 44, (o) SEQ ID NOS:45 and 46, (p) SEQ ID NOS:47 and 48, (q) SEQ ID NOS:49 and 50, (r) SEQ ID NOS:51 and 52, (s) SEQ ID NOS:53 and 54, (t) SEQ ID NOS:55 and 56, (u) SEQ ID NOS:57 and 58, (v) SEQ ID NOS:59 and 60, and (w) SEQ ID NOS:61 and 62.

30. A method of inhibiting differentiation of a pre-adipocyte to an adipocyte, said method comprising contacting said pre-adipocyte with (i) a short, interfering (si) RNA from 16 to 29 nucleotides in length, said siRNA having a first strand and a second strand, each strand being (a) of equal length, (b) from 16 to 29 nucleotides in length, (c) hybridized to the other strand, and (d) having a 5′ end and a 3′ end, and wherein in which the 16 to 29 nucleotides of one strand are complementary to a corresponding length of galectin-12 mRNA (SEQ ID NO:1), (ii) a vector encoding a siRNA of (i), or (iii) both (i) and (ii), thereby inhibiting activity of galectin-12 in said pre-adipocyte.

31. A method of claim 30, further wherein said first strand and said second strand have at least one unpaired nucleotide on their respective 3′ ends.

32. A method of claim 30, wherein one of said two strands has a sequence selected from the group consisting of SEQ ID NO:9, SEQ ID NO:10, SEQ ID NO:11, SEQ ID NO:12, SEQ ID NO:13, SEQ ID NO:14, SEQ ID NO:21, SEQ ID NO:22, SEQ ID NO:23, SEQ ID NO:24, SEQ ID NO:25, and SEQ ID NO:26.

33. A method of claim 30, wherein said strands are selected from the group consisting of (a) SEQ ID NOS:3 and 4, (b) SEQ ID NOS:5 and 6, (c) SEQ ID NOS:7 and 8, (d) SEQ ID NOS:9 and 10, (e) SEQ ID NOS:11 and 12, (f) SEQ ID NOS:13 and 14, (g) SEQ ID NOS:15 and 16, (h) SEQ ID NOS:31 and 32, (i) SEQ ID NOS:33 and 34, (j) SEQ ID NOS:35 and 36, (k) SEQ ID NOS:37 and 38, (l) SEQ ID NOS:39 and 40, (m) SEQ ID NOS:41 and 42, (n) SEQ ID NOS:43 and 44, (o) SEQ ID NOS:45 and 46, (p) SEQ ID NOS:47 and 48, (q) SEQ ID NOS:49 and 50, (r) SEQ ID NOS:51 and 52, (s) SEQ ID NOS:53 and 54, (t) SEQ ID NOS:55 and 56, (u) SEQ ID NOS:57 and 58, (v) SEQ ID NOS:59 and 60, and (w) SEQ ID NOS:61 and 62.

34. A method of inhibiting differentiation of pre-adipocytes into adipocytes, said method comprising administering an inhibitor of galectin-12 activity, thereby inhibiting said differentiation of pre-adipocytes.

35. A method of inhibiting differentiation of leukocytes, said method comprising administering an inhibitor of galectin-12 activity, thereby inhibiting said differentiation of leukocytes.

36. A kit comprising
(a) a container and
(b) (i) a short, interfering (si) RNA from 16 to 29 nucleotides in length, said siRNA having a first strand and a second strand, each strand being (A) of equal length, (B) from 16 to 29 nucleotides in length, (C) hybridized to the other strand, and (D) having a 5′ end and a 3′ end, and wherein in which the 16 to 29 nucleotides of one strand are complementary to a corresponding length of galectin-12 mRNA (SEQ ID NO:1), or
(ii) a vector encoding a siRNA of (b)(i), or
(iii) both (b)(i) and (b)(ii).

37. A kit of claim 36, further comprising a pharmaceutically acceptable carrier.


SEQUENCE LISTING (85 sequences)

SEQ ID NO: 1 - DNA, 1696 bp, Homo sapiens
  human galectin-12 (lectin, galactoside-binding, soluble, 12 (LGALS12))
acaaaccctcgttggccccacagggagccagcctctggcttctctctgcaatggccatgtgctgcagacccggagtgggtagttagttggttaatgccagtcttcctcccctggacactgagttctgctgacagcccccgcccagccagagctctgctgtataccaccgggagtggggctggtgtggagcctggaggtcgcccgctgccctcctagggctgctccagacagcattaaaacgctgcaggtcgcaggtgagactaacagctgggagagctgctccaggcatttaggaccctgactggggcagatgagtcagcccagtgggggcagggctcctggaacgaggatctacagttggagttgccccactgtcatgtcacctggagaaaaactggacccaattcctgacagcttcattctgcaaccaccagtcttccacccggtggttccttatgtcacgacgatttttggaggcctgcatgcaggcaagatggtcatgctgcaaggagtggtccctctagatgcacacaggtttcaggtggacttccagtgtggctgcagcctgtgtccccggccagatatcgccttccacttcaaccctcgcttccataccaccaagccccatgtcatctgcaacaccctgcatggtggacgctggcaaagggaggcccggtggccccacctggccctgcgaagaggctccagcttcctcatcctctttctcttcgggaatgaggaagtgaaggtgagtgtgaatggacagcactttctccacttccgctaccggctcccactgtctcatgtggacacgctgggtatatttggtgacatcctggtagaggctgttggattcctgaacatcaatccatttgtggagggcagcagagagtacccagctggacatcctttcctgctgatgagccccaggctggaggtgccctgctcacatgctcttccccagggtctctcgcctgggcaggtcatcatagtacggggactggtcttgcaagagccgaagcattttactgtgagcctgagggaccaggctgcccatgctcctgtgacactcagggcctccttcgcagacagaactctggcctggatctcccgctgggggcagaagaaactgatctcagcccccttcctcttttacccccagagattctttgaggtgctgctcctgttccaggagggagggctgaagctggcgctcaatgggcaggggctgggggccaccagcatgaaccagcaggccctggagcagctgcgggagctccggatcagtggaagtgtccagctctactgtgtccactcctgaggatggttccaggggaaataccgccagaaaacaagaaggtcagcccactcccagggccccactctcctcccctcattaaaccatccacctgacaccagcacatcaggcctggttcacctctggggtcacgagactgagtctacaggagctttgggcctgagggaaggcacaagagtgcaaaggttcctcgaactctgcaccttcctccaccaggagcctgggatatggctccatctgccttcagggcctggactgcactcacagaggcaagtgttgtagactaacaaagatactccaaaatacaatggcttaaagaatgtggtcatttattctttattatttatttatttgtggtcaaataaataaataaggtta

SEQ ID NO: 2 - protein, 336 aa, Homo sapiens
  human galectin-12 (lectin, galactoside-binding, soluble, 12 (LGALS12))
MSQPSGGRAPGTRIYSWSCPTVMSPGEKLDPIPDSFILQPPVFHPVVPYVTTIFGGLHAGKMVMLQGVVPLDAHRFQVDFQCGCSLCPRPDIAFHFNPRFHTTKPHVICNTLHGGRWQREARWPHLALRRGSSFLILFLFGNEEVKVSVNGQHFLHFRYRLPLSHVDTLGIFGDILVEAVGFLNINPFVEGSREYPAGHPFLLMSPRLEVPCSHALPQGLSPGQVIIVRGLVLQEPKHFTVSLRDQAAHAPVTLRASFADRTLAWISRWGQKKLISAPFLFYPQRFFEVLLLFQEGGLKLALNGQGLGATSMNQQALEQLRELRISGSVQLYCVHS

SEQ ID NO: 3 - RNA, 21 nt, Artificial Sequence
  Description of Artificial Sequenceshort or small interfering RNA (siRNA) predicted from human galectin-12 mRNA
gauaucgccuuccacuucauu

SEQ ID NO: 4 - RNA, 21 nt, Artificial Sequence
  Description of Artificial Sequenceshort or small interfering RNA (siRNA) predicted from human galectin-12 mRNA
ugaaguggaaggcgauaucuu

SEQ ID NO: 5 - RNA, 21 nt, Artificial Sequence
  Description of Artificial Sequenceshort or small interfering RNA (siRNA) from mouse galectin-12 mRNA with 100% identity to human galectin-12 mRNA
uuccugaacaucaauccauuu

SEQ ID NO: 6 - RNA, 21 nt, Artificial Sequence
  Description of Artificial Sequenceshort or small interfering RNA (siRNA) from mouse galectin-12 mRNA with 100% identity to human galectin-12 mRNA
auggauugauguucaggaauu

SEQ ID NO: 7 - RNA, 21 nt, Artificial Sequence
  Description of Artificial Sequenceshort or small interfering RNA (siRNA) from mouse galectin-12 mRNA with 100% identity to human galectin-12 mRNA
caucaauccauuuguggaguu

SEQ ID NO: 8 - RNA, 21 nt, Artificial Sequence
  Description of Artificial Sequenceshort or small interfering RNA (siRNA) from mouse galectin-12 mRNA with 100% identity to human galectin-12 mRNA
cuccacaaauggauugauguu

SEQ ID NO: 9 - RNA, 21 nt, Artificial Sequence
  Description of Artificial Sequenceshort or small interfering RNA (siRNA) predicted from human galectin-12 mRNA including 3′ untranslated regions and using less stringent criteria
caagagugcaaagguuccuuu

SEQ ID NO: 10 - RNA, 21 nt, Artificial Sequence
  Description of Artificial Sequenceshort or small interfering RNA (siRNA) predicted from human galectin-12 mRNA including 3′ untranslated regions and using less stringent criteria
aggaaccuuugcacucuuguu

SEQ ID NO: 11 - RNA, 21 nt, Artificial Sequence
  Description of Artificial Sequenceshort or small interfering RNA (siRNA) predicted from human galectin-12 mRNA including 3′ untranslated regions and using less stringent criteria
gaggcaaguguuguagacuuu

SEQ ID NO: 12 - RNA, 21 nt, Artificial Sequence
  Description of Artificial Sequenceshort or small interfering RNA (siRNA) predicted from human galectin-12 mRNA including 3′ untranslated regions and using less stringent criteria
agucuacaacacuugccucuu

SEQ ID NO: 13 - RNA, 21 nt, Artificial Sequence
  Description of Artificial Sequenceshort or small interfering RNA (siRNA) predicted from human galectin-12 mRNA including 3′ untranslated regions and using less stringent criteria
ggcaaguguuguagacuaauu

SEQ ID NO: 14 - RNA, 21 nt, Artificial Sequence
  Description of Artificial Sequenceshort or small interfering RNA (siRNA) predicted from human galectin-12 mRNA including 3′ untranslated regions and using less stringent criteria
uuagucuacaacacuugccuu

SEQ ID NO: 15 - RNA, 21 nt, Artificial Sequence
  Description of Artificial Sequenceshort or small interfering RNA (siRNA) predicted from human galectin-12 mRNA including 3′ untranslated regions and using less stringent criteria
auacaauggcuuaaagaauuu

SEQ ID NO: 16 - RNA, 21 nt, Artificial Sequence
  Description of Artificial Sequenceshort or small interfering RNA (siRNA) predicted from human galectin-12 mRNA including 3′ untranslated regions and using less stringent criteria
auucuuuaagccauuguauuu

SEQ ID NO: 17 - RNA, 19 nt, Artificial Sequence
  Description of Artificial Sequenceshort or small interfering RNA (siRNA) without two-nucleotide 3′-overhang
gauaucgccuuccacuuca

SEQ ID NO: 18 - RNA, 19 nt, Artificial Sequence
  Description of Artificial Sequenceshort or small interfering RNA (siRNA) without two-nucleotide 3′-overhang
ugaaguggaaggcgauauc

SEQ ID NO: 19 - RNA, 19 nt, Artificial Sequence
  Description of Artificial Sequenceshort or small interfering RNA (siRNA) without two-nucleotide 3′-overhang
uuccugaacaucaauccau

SEQ ID NO: 20 - RNA, 19 nt, Artificial Sequence
  Description of Artificial Sequenceshort or small interfering RNA (siRNA) without two-nucleotide 3′-overhang
auggauugauguucaggaa

SEQ ID NO: 21 - RNA, 19 nt, Artificial Sequence
  Description of Artificial Sequenceshort or small interfering RNA (siRNA) without two-nucleotide 3′-overhang
caucaauccauuuguggag

SEQ ID NO: 22 - RNA, 19 nt, Artificial Sequence
  Description of Artificial Sequenceshort or small interfering RNA (siRNA) without two-nucleotide 3′-overhang
cuccacaaauggauugaug

SEQ ID NO: 23 - RNA, 19 nt, Artificial Sequence
  Description of Artificial Sequenceshort or small interfering RNA (siRNA) without two-nucleotide 3′-overhang
caagagugcaaagguuccu

SEQ ID NO: 24 - RNA, 19 nt, Artificial Sequence
  Description of Artificial Sequenceshort or small interfering RNA (siRNA) without two-nucleotide 3′-overhang
aggaaccuuugcacucuug

SEQ ID NO: 25 - RNA, 19 nt, Artificial Sequence
  Description of Artificial Sequenceshort or small interfering RNA (siRNA) without two-nucleotide 3′-overhang
gaggcaaguguuguagacu

SEQ ID NO: 26 - RNA, 19 nt, Artificial Sequence
  Description of Artificial Sequenceshort or small interfering RNA (siRNA) without two-nucleotide 3′-overhang
agucuacaacacuugccuc

SEQ ID NO: 27 - RNA, 19 nt, Artificial Sequence
  Description of Artificial Sequenceshort or small interfering RNA (siRNA) without two-nucleotide 3′-overhang
ggcaaguguuguagacuaa

SEQ ID NO: 28 - RNA, 19 nt, Artificial Sequence
  Description of Artificial Sequenceshort or small interfering RNA (siRNA) without two-nucleotide 3′-overhang
uuagucuacaacacuugcc

SEQ ID NO: 29 - RNA, 19 nt, Artificial Sequence
  Description of Artificial Sequenceshort or small interfering RNA (siRNA) without two-nucleotide 3′-overhang
auacaauggcuuaaagaau

SEQ ID NO: 30 - RNA, 19 nt, Artificial Sequence
  Description of Artificial Sequenceshort or small interfering RNA (siRNA) without two-nucleotide 3′-overhang
auucuuuaagccauuguau

SEQ ID NO: 31 - DNA, 21 bp, Artificial Sequence
  Description of Combined DNA/RNA Moleculelonger short or small interfering RNA (siRNA) nucleotide duplexes with deoxyribonucleotide two-nucleotide 3′-overhang replacements
gauaucgccuuccacuucatt

SEQ ID NO: 32 - DNA, 21 bp, Artificial Sequence
  Description of Combined DNA/RNA Moleculelonger short or small interfering RNA (siRNA) nucleotide duplexes with deoxyribonucleotide two-nucleotide 3′-overhang replacements
ugaaguggaaggcgauauctt

SEQ ID NO: 33 - DNA, 21 bp, Artificial Sequence
  Description of Combined DNA/RNA Moleculelonger short or small interfering RNA (siRNA) nucleotide duplexes with deoxyribonucleotide two-nucleotide 3′-overhang replacements
uuccugaacaucaauccautt

SEQ ID NO: 34 - DNA, 21 bp, Artificial Sequence
  Description of Combined DNA/RNA Moleculelonger short or small interfering RNA (siRNA) nucleotide duplexes with deoxyribonucleotide two-nucleotide 3′-overhang replacements
auggauugauguucaggaatt

SEQ ID NO: 35 - DNA, 21 bp, Artificial Sequence
  Description of Combined DNA/RNA Moleculelonger short or small interfering RNA (siRNA) nucleotide duplexes with deoxyribonucleotide two-nucleotide 3′-overhang replacements
caucaauccauuuguggagtt

SEQ ID NO: 36 - DNA, 21 bp, Artificial Sequence
  Description of Combined DNA/RNA Moleculelonger short or small interfering RNA (siRNA) nucleotide duplexes with deoxyribonucleotide two-nucleotide 3′-overhang replacements
cuccacaaauggauugaugtt

SEQ ID NO: 37 - RNA, 31 nt, Artificial Sequence
  Description of Artificial Sequenceshort or small interfering RNA (siRNA) nucleotide duplexes longer than 19-mer by extending target-complementary strand
gauaucgccuuccacuucaacccucgcuuuu

SEQ ID NO: 38 - RNA, 31 nt, Artificial Sequence
  Description of Artificial Sequenceshort or small interfering RNA (siRNA) nucleotide duplexes longer than 19-mer by extending target-complementary strand
aagcgaggguugaaguggaaggcgauaucuu

SEQ ID NO: 39 - RNA, 31 nt, Artificial Sequence
  Description of Artificial Sequenceshort or small interfering RNA (siRNA) nucleotide duplexes longer than 19-mer by extending target-complementary strand
uuccugaacaucaauccauuuguggaggguu

SEQ ID NO: 40 - RNA, 31 nt, Artificial Sequence
  Description of Artificial Sequenceshort or small interfering RNA (siRNA) nucleotide duplexes longer than 19-mer by extending target-complementary strand
cccuccacaaauggauugauguucaggaauu

SEQ ID NO: 41 - RNA, 31 nt, Artificial Sequence
  Description of Artificial Sequenceshort or small interfering RNA (siRNA) nucleotide duplexes longer than 19-mer by extending target-complementary strand
caucaauccauuuguggagggcagcagaguu

SEQ ID NO: 42 - RNA, 31 nt, Artificial Sequence
  Description of Artificial Sequenceshort or small interfering RNA (siRNA) nucleotide duplexes longer than 19-mer by extending target-complementary strand
cucugcugcccuccacaaauggauugauguu

SEQ ID NO: 43 - RNA, 30 nt, Artificial Sequence
  Description of Artificial Sequenceshort or small interfering RNA (siRNA) nucleotide duplexes longer than 19-mer by extending target-complementary strand
gauaucgccuuccacuucaacccucgcuuu

SEQ ID NO: 44 - RNA, 30 nt, Artificial Sequence
  Description of Artificial Sequenceshort or small interfering RNA (siRNA) nucleotide duplexes longer than 19-mer by extending target-complementary strand
agcgaggguugaaguggaaggcgauaucuu

SEQ ID NO: 45 - RNA, 28 nt, Artificial Sequence
  Description of Artificial Sequenceshort or small interfering RNA (siRNA) nucleotide duplexes longer than 19-mer by extending target-complementary strand
uuccugaacaucaauccauuuguggauu

SEQ ID NO: 46 - RNA, 28 nt, Artificial Sequence
  Description of Artificial Sequenceshort or small interfering RNA (siRNA) nucleotide duplexes longer than 19-mer by extending target-complementary strand
uccacaaauggauugauguucaggaauu

SEQ ID NO: 47 - RNA, 27 nt, Artificial Sequence
  Description of Artificial Sequenceshort or small interfering RNA (siRNA) nucleotide duplexes longer than 19-mer by extending target-complementary strand
caucaauccauuuguggagggcagcuu

SEQ ID NO: 48 - RNA, 27 nt, Artificial Sequence
  Description of Artificial Sequenceshort or small interfering RNA (siRNA) nucleotide duplexes longer than 19-mer by extending target-complementary strand
gcugcccuccacaaauggauugauguu

SEQ ID NO: 49 - RNA, 23 nt, Artificial Sequence
  Description of Artificial Sequenceshort or small interfering RNA (siRNA) nucleotide duplexes longer than 19-mer by extending target-complementary strand
gauaucgccuuccacuucaacuu

SEQ ID NO: 50 - RNA, 23 nt, Artificial Sequence
  Description of Artificial Sequenceshort or small interfering RNA (siRNA) nucleotide duplexes longer than 19-mer by extending target-complementary strand
guugaaguggaaggcgauaucuu

SEQ ID NO: 51 - RNA, 24 nt, Artificial Sequence
  Description of Artificial Sequenceshort or small interfering RNA (siRNA) nucleotide duplexes longer than 19-mer by extending target-complementary strand
uuccugaacaucaauccauuuguu

SEQ ID NO: 52 - RNA, 24 nt, Artificial Sequence
  Description of Artificial Sequenceshort or small interfering RNA (siRNA) nucleotide duplexes longer than 19-mer by extending target-complementary strand
caaauggauugauguucaggaauu

SEQ ID NO: 53 - RNA, 24 nt, Artificial Sequence
  Description of Artificial Sequenceshort or small interfering RNA (siRNA) nucleotide duplexes longer than 19-mer by extending target-complementary strand
caucaauccauuuguggagggcuu

SEQ ID NO: 54 - RNA, 24 nt, Artificial Sequence
  Description of Artificial Sequenceshort or small interfering RNA (siRNA) nucleotide duplexes longer than 19-mer by extending target-complementary strand
gcccuccacaaauggauugauguu

SEQ ID NO: 55 - RNA, 18 nt, Artificial Sequence
  Description of Artificial Sequenceshort or small interfering RNA (siRNA) 18-mer duplex form
gauaucgccuuccacuuc

SEQ ID NO: 56 - RNA, 18 nt, Artificial Sequence
  Description of Artificial Sequenceshort or small interfering RNA (siRNA) 18-mer duplex form
gaaguggaaggcgauauc

SEQ ID NO: 57 - RNA, 17 nt, Artificial Sequence
  Description of Artificial Sequenceshort or small interfering RNA (siRNA) 17-mer duplex form
gauaucgccuuccacuu

SEQ ID NO: 58 - RNA, 17 nt, Artificial Sequence
  Description of Artificial Sequenceshort or small interfering RNA (siRNA) 17-mer duplex form
aaguggaaggcgauauc

SEQ ID NO: 59 - RNA, 16 nt, Artificial Sequence
  Description of Artificial Sequenceshort or small interfering RNA (siRNA) 16-mer duplex form
gauaucgccuuccacu

SEQ ID NO: 60 - RNA, 16 nt, Artificial Sequence
  Description of Artificial Sequenceshort or small interfering RNA (siRNA) 16-mer duplex form
aguggaaggcgauauc

SEQ ID NO: 61 - RNA, 20 nt, Artificial Sequence
  Description of Artificial Sequenceshort or small interfering RNA (siRNA) 18-mer duplex form with 3′ overhang
gauaucgccuuccacuucuu

SEQ ID NO: 62 - RNA, 20 nt, Artificial Sequence
  Description of Artificial Sequenceshort or small interfering RNA (siRNA) 18-mer duplex form with 3′ overhang
gaaguggaaggcgauaucuu

SEQ ID NO: 63 - RNA, 21 nt, Artificial Sequence
  Description of Artificial Sequencecontrol short or small interfering RNA (siRNA) siGL2 with American firefly (Photinus pyralis) luciferase mRNA target
aacguacgcggaauacuucga

SEQ ID NO: 64 - RNA, 21 nt, Artificial Sequence
  Description of Artificial Sequencecontrol short or small interfering RNA (siRNA) si3 with mouse galectin-3 mRNA target
aaacaggauuguucuagauuu

SEQ ID NO: 65 - RNA, 21 nt, Artificial Sequence
  Description of Artificial Sequenceshort or small interfering RNA (siRNA) si12.1 with mouse galectin-12 mRNA target
aauuccugaacaucaauccau

SEQ ID NO: 66 - RNA, 21 nt, Artificial Sequence
  Description of Artificial Sequenceshort or small interfering RNA (siRNA) si12.2 with mouse galectin-12 mRNA target
aacaucaauccauuuguggag

SEQ ID NO: 67 - RNA, 21 nt, Artificial Sequence
  Description of Artificial Sequenceshort or small interfering RNA (siRNA) si12.3 with mouse galectin-12 mRNA target
aaucuggugacaucuugguaa

SEQ ID NO: 68 - DNA, 22 bp, Artificial Sequence
  Description of Artificial SequenceRT-PCR primer for mouse galectin-12
cctgctcacgtgctcttcctcg

SEQ ID NO: 69 - DNA, 22 bp, Artificial Sequence
  Description of Artificial SequenceRT-PCR primer for mouse galectin-12
ttggagcccttcttagcagtgg

SEQ ID NO: 70 - DNA, 26 bp, Artificial Sequence
  Description of Artificial SequenceRT-PCR primer for mouse GAPDH control
tgaaggtcggtgtgaacggatttggc

SEQ ID NO: 71 - DNA, 24 bp, Artificial Sequence
  Description of Artificial SequenceRT-PCR primer for mouse GAPDH control
catgtaggccatgaggtccaccac

SEQ ID NO: 72 - DNA, 2468 bp, Mus musculus
  mouse galectin-12 cDNA
ggcacgagggcagcagcttcccttcctactatccgctgaaggcccagctgttgcagaggctgggaaaagtgagtccattccgtggggaatcgcgttgctggaatcaggatcagaggctgagttgaagttgcccctcccccatgtcaactgacgaacacctggacccgattcctgacagcttcatcctgcagccgccagtcttccacccggtgattccttatggcacaacaatttttggtggcctgtatgcaggcaagatggtcacgctgcagggtgtggtccctctgcatgcaaggaggtttcaggtggacttccagtgtgggtgctgcctgcatcctcagccagatgttgccttccgcttcagccctcgattctacactgtcaagccccatgtcatctgcaacacccaccaaggtggactctggcaaaaagagatccggtggccaggggtcgccctgcagagaggggatagcttcctcattctctttctctttgagaacgaagaggtgaaggtgagtgtaaatggccagcactttcttcactaccgctaccggctcccactgtcacgggtagatacccttgacatatctggtgacatcttggtaaaggctgttggattcctgaacatcaatccatttgtggagggtagcagagagtatccagttggatatcccttcctgctgtatagccccaggctggaggtgccctgctcacgtgctcttcctcggggtctctggcctgggcaagtcattgtagttcgaggactggtcttgaaagagccgaaagattttaccctgagcctgaaggatgggaccacccatgttcctgtgacactcagggcttccttcacagacagaacactggcctgggtctcctcctggggacgaaagaagctgatctccgcccccttcctctttcacccccagcgattcttcgaggtactgcttctgtgccaagagggagggctgaagttggcactcaatgggcaggggctgggggccaccagcctggaccagaaagccctggagcagctgcgggagctcaggatcagtggaaatgtccacctctactgtgtccactgctaagaagggctccaagggcacccccgccagagaagaggaggggcggctgcaaccagggctccacagttacagcccattctcctattctcaagggagaaactgacctggcatcactttgtcaggccgagctcacatctcaaagggcgcgcgcacacacacacacacacacacacacacacacacacacacacacacacacattgcattttaatctatcaattctgaaaataagggcccaggtcaatgtgtctctgcatctatcttccacattcaaattctgtcttggtcttcattccaatccaggcccagccactgagaaggcaaatagagtgtgaacaagatgagccccaaatctgtgatcaacacacaccatctacactcaccttttcatctacacttgttttatctacagtcactcactagtttgtgaagcctttgcccaaagtgaacaaaatatcacagctgctttcatggttttggccaaagggaaccagccacatcctaggaaaacacactgattatgggaccttttggtcacccaaccatgtaaacaacaataatctctcataaaattaatctataatatagtctgggtgggccagggattattcagtctatgtttctacagcataccctgcaatatctatacaatggccaaatggtcaaacaatgcatttcccagaatttgtccttgtcatgaaatgatacatgactgtatctagcagagcaggtagaccaggaaaaaggaagctgtagagtgtttgatttgaactattcctggaaatgaagtctgggctcactttaaatctaaagtgacttaagactctgtctcctgggaatgtttctgtaatcccatctacctgcccagagctgtgcccgggtacacactgtagaagatactgatgaaggatttgctgtgttctaacattatactctaagcaaagagccgttttcttcccttgtccatttaccaagattgatcatgtacctttcataaaggaaaaaaaaaccctcaaatttccaaggggaaagtttttactgcaaatatatgtctttattgtaatccagggaaactagaggtcagaaaaagaggtggctgagaggcatcccctagacaccaaagcccggaactctaggaagaatctatgtcaaagaagagatggcaggttggtggttgaggttacttagttaaccttctaatgaaataaactatattgagaatctctaatatatatcttagtatactatactaaatttagggaataaaatttatttggtgttgattaatcttaataaaaatacagtgtacattttatccactgatattatgtatcttatgttcataaattaaattggcttgtcattctaaaaaaaaaaaaaaaaaaa

SEQ ID NO: 73 - protein, 314 aa, Homo sapiens
  human galectin-12 (LEG12) positions 23-336
MSPGEKLDPIPDSFILQPPVFHPVVPYVTTIFGGLHAGKMVMLQGVVPLDAHRFQVDFQCGCSLCPRPDIAFHFNPRFHTTKPHVICNTLHGGRWQREARWPHLALRRGSSFLILFLFGNEEVKVSVNGQHFLHFRYRLPLSHVDTLGIFGDILVEAVGFLNINPFVEGSREYPAGHPFLLMSPRLEVPCSHALPQGLSPGQVIIVRGLVLQEPKHFTVSLRDQAAHAPVTLRASFADRTLAWISRWGQKKLISAPFLFYPQRFFEVLLLFQEGGLKLALNGQGLGATSMNQQALEQLRELRISGSVQLYCVHS

SEQ ID NO: 74 - protein, 314 aa, Mus musculus
  mouse galectin-12 (LEG12)
MSTDEHLDPIPDSFILQPPVFHPVIPYGTTIFGGLYAGKMVTLQGVVPLHARRFQVDFQCGCCLHPQPDVAFRFSPRFYTVKPHVICNTHQGGLWQKEIRWPGVALQRGDSFLILFLFENEEVKVSVNGQHFLHYRYRLPLSRVDTLDISGDILVKAVGFLNINPFVEGSREYPVGYPFLLYSPRLEVPCSRALPRGLWPGQVIVVRGLVLKEPKDFTLSLKDGTTHVPVTLRASFTDRTLAWVSSWGRKKLISAPFLFHPQRFFEVLLLCQEGGLKLALNGQGLGATSLDQKALEQLRELRISGNVHLYCVHC

SEQ ID NO: 75 - protein, 301 aa, Mus musculus
  mouse galectin-6 (LEG6)
MAYVPAPGYQPTYNPTLPYKRPIPGGLSVGMSFYIQGTAKENMRRFHVNFAVGQDDGADVAFHFNPRFDGWDKVVFNTKQSGRWGKEEEKSMPFQKGKHFELVFMVMPEHYKVVVNGSPFYEYGHRLPVQMVTHLQVDGDLELQSINFFGVQPAETKYPAMTGPPVFNPCLPYVGALQGGFTVRRTIIIKGYVLPTAKTFAINFRVGSSEDIALHINPRIGDCLVRNSYMNGSWGTEERMVAYNPFGPGQFFDLSIRCGMDRFKVFANGIHLFNFSHRFQALRKINTLEINGDLTLSYVHI

SEQ ID NO: 76 - protein, 323 aa, Homo sapiens
  human galectin-4 (LEG4)
MAYVPAPGYQPTYNPTLPYYQPIPGGLNVGMSVYIQGVASEHMKRFFVNFVVGQDPGSDVAFHFNPRFDGWDKVVFNTLQGGKWGSEERKRSMPFKKGAAFELVFIVLAEHYKVVVNGNPFYEYGHRLPLQMVTHLQVDGDLQLQSINFIGGQPLRPQGPPMMPPYPGPGHCHQQLNSLPTMEGPPTFNPPVPYFGRLQGGLTARRTIIIKGYVPPTGKSFAINFKVGSSGDIALHINPRMGNGTVVRNSLLNGSWGSEEKKITHNPFGPGQFFDLSIRCGLDRFKVYANGQHLFDFAHRLSAFQRVDTLEIQGDVTLSYVQI

SEQ ID NO: 77 - protein, 316 aa, Homo sapiens [sequence omitted]

SEQ ID NO: 78 - protein, 355 aa, Homo sapiens
  human galectin-9 (LEG9)
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 79 - RNA, 21 nt, Artificial Sequence
  Description of Artificial Sequenceshort or small interfering RNA (siRNA) siGL2
cguacgcggaauacuucgauu

SEQ ID NO: 80 - RNA, 21 nt, Artificial Sequence
  Description of Artificial Sequenceshort or small interfering RNA (siRNA) siGL2
ucgaaguauuccgcguacguu

SEQ ID NO: 81 - RNA, 21 nt, Artificial Sequence
  Description of Artificial Sequenceshort or small interfering RNA (siRNA) si3
acaggauuguucuagauuuuu

SEQ ID NO: 82 - RNA, 21 nt, Artificial Sequence
  Description of Artificial Sequenceshort or small interfering RNA (siRNA) si3
aaauauagaacaauccuguuu

SEQ ID NO: 83 - RNA, 21 nt, Artificial Sequence
  Description of Artificial Sequenceshort or small interfering RNA (siRNA) si12.3
ucuggugacaucuugguaauu

SEQ ID NO: 84 - RNA, 21 nt, Artificial Sequence
  Description of Artificial Sequenceshort or small interfering RNA (siRNA) si12.3
uuaccaagaugucaccagauu

SEQ ID NO: 85 - DNA, 1647 bp, Homo sapiens
  human galectin-12 cDNA from GenBank Accession #BC028222
cacaggagccagcctctggcttctctctgcaatggccatgtgctgcagacccggagtgggtagttagttggttaatgccagtcttcctcccctggacactgagttctgctgacagcccccgcccagccagagctctgctgtataccaccgggagtggggctggtgtggagcctggaggtcgcccgctgccctcctagggctgctccagacagcattaaaacgctgcaggtcgcaggtgagactaacagctgggagagctgctccaggcatttaggaccctgactggggcagatgagtcagcccagtgggggcagggctcctggaacgaggatctacagttggagttgccccactgtcatgtcacctggagaaaaactggacccaattcctgacagcttcattctgcaaccaccagtcttccacccggtggttccttatgtcacgacgatttttggaggcctgcatgcaggcaagatggtcatgctgcaaggagtggtccctctagatgcacacaggtttcaggtggacttccagtgtggctgcagcctgtgtccccggccagatatcgccttccacttcaaccctcgcttccataccaccaagccccatgtcatctgcaacaccctgcatggtggacgctggcaaagggaggcccggtggccccacctggccctgcgaagaggctccagcttcctcatcctctttctcttcgggaatgaggaagtgaaggtgagtgtgaatggacagcactttctccacttccgctaccggctcccactgtctcatgtggacacgctgggtatatttggtgacatcctggtagaggctgttggattcctgaacatcaatccatttgtggagggcagcagagagtacccagctggacatcctttcctgctgatgagccccaggctggaggtgccctgctcacatgctcttccccagggtctctcgcctgggcaggtcatcatagtacggggactggtcttgcaagagccgaagcattttactgtgagcctgagggaccaggctgcccatgctcctgtgacactcagggcctccttcgcagacagaactctggcctggatctcccgctgggggcagaagaaactgatctcagcccccttcctcttttacccccagagattctttgaggtgctgctcctgttccaggagggagggctgaagctggcgctcaatgggcaggggctgggggccaccagcatgaaccagcaggccctggagcagctgcgggagctccggatcagtggaagtgtccagctctactgtgtccactcctgaggatggttccagggaaataccgccagaaaacaagaaggtcagcccactcccagggccccactctcctcccctcattaaaccatccacctgacaccagcacatcaggcctggttcacctctggggtcacgagactgagtctacaggagctttgggcctgagggaaggcacaagagtgcaaaggttcctcgaactctgcaccttcctccaccaggagcctgggatatggctccatctgccttcagggcctggactgcactcacagaggcaagtgttgtagactaacaaagatactccaaaatacaatggcttaaagaaaaaaaaaaaaaaaaaaaaaaaaaaaaaaa